(19) United States
(12) Patent Application Publication
(10) Pub. No.: US 2024/0277705 A1
(43) Pub. Date: Aug. 22, 2024

(54) DOSAGE REGIMENS FOR ECUBECTEDIN

(71) Applicant: PHARMA MAR S.A., Madrid (ES)

(72) Inventors: Carmen Kahatt, Madrid (ES); Pilar Lardelli, Madrid (ES); Cristian Fernandez, Madrid (ES); Arturo Soto, Madrid (ES)

(21) Appl. No.: 18/560,991

(22) PCT Filed: May 19, 2022
(86) PCT No.: PCT/EP2022/063653

§ 371 Date: Nov. 15, 2023

(30) Foreign Application Priority Data
May 19, 2021 (EP) 21382455.0

(51) Int. Cl. A61K 31/4985; A61P 35/00

(52) U.S. Cl. CPC A61K 31/4985; A61P 35/00

(57) ABSTRACT

The present invention relates to a compound of formula (I), or a pharmaceutically acceptable salt or ester thereof, for use in the treatment of cancers. The present invention also relates to a compound of formula I or a pharmaceutically acceptable salt or ester thereof, for use in the treatment of cancer at certain dosage regimens.

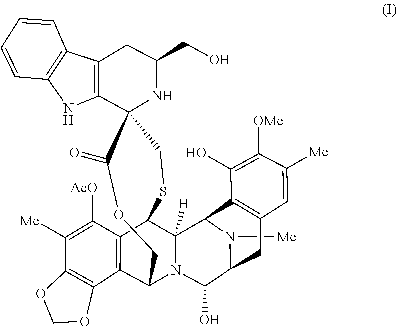 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

### FIELD OF THE INVENTION

The present invention relates to an ecteinascidin compound (PM14) for use in the treatment of certain cancers. The present invention also relates to dosage regimens of PM14 for use in the treatment of cancer.

### BACKGROUND TO THE INVENTION

The ecteinascidins are exceedingly potent antitumor agents isolated from the marine tunicate Ecteinascidia turbinata. WO2018/197663 describes synthetic ecteinascidin compounds including PM14 which is described as compound 4-S with the following formula:

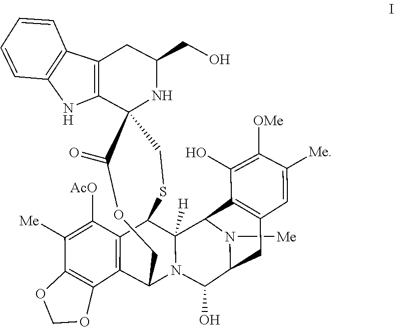 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

PM14 was shown in WO2018/197663 to demonstrate in vitro activity against non-small cell lung cancer (NSCLC), colorectal adenocarcinoma, breast adenocarcinoma, pancreas adenocarcinoma, prostate adenocarcinoma, and prostate carcinoma cell lines and in vivo activity in fibrosarcoma, breast adenocarcinoma, NSCLC, ovarian carcinoma, gastric carcinoma, small cell lung cancer (SCLC), prostatic adenocarcinoma, and prostatic carcinoma xenograph models.

There remains a need to develop new treatments of cancer(s) and/or improved treatments of cancer(s). The present invention addresses this need.

### SUMMARY OF THE INVENTION

According to an aspect of the present invention, there is provided a compound of formula I:

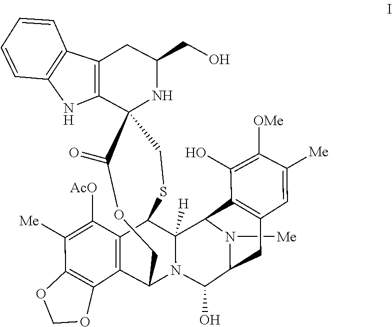 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

or a pharmaceutically acceptable salt or ester thereof, for use in the treatment of cancer, wherein the compound is administered to a subject in a three-week cycle at a total dose from about 0.5 mg/m² to about 9 mg/m², preferably from about 1.0 mg/m² to about 9.0 mg/m², about 1.5 mg/m² to about 9.0 mg/m², about 2.0 mg/m² to about 9.0 mg/m², about 2.5 mg/m² to about 8.5 mg/m², about 3.0 mg/m² to about 8.0 mg/m², about 3.5 mg/m² to about 7.5 mg/m², about 4.0 mg/m² to about 7.0 mg/m², about 4.0 mg/m² to about 6.5 mg/m², about 4.5 mg/m² to about 6.5 mg/m², about 4.5 mg/m² to about 6.0 mg/m^{2.}

For the first time, clinical dosage regimens have been identified which are well tolerated and have a manageable safety profile. In addition, efficacy in humans has been demonstrated in relation to the dosage regimen.

The total dose may be about 3.0 mg/m² to about 6.0 mg/m², about 3.0 mg/m² to about 5.6 mg/m², about 3.5 mg/m² to about 5.6 mg/m², about 4.0 mg/m² to about 5.0 mg/m² or about 4.5 mg/m^{2.}

The total dose may be about 4.0 mg/m² to about 9.0 mg/m², about 4.0 mg/m² to about 8.0 mg/m², about 4.5 mg/m² to about 7.5 mg/m², about 5.0 mg/m² to about 7.0 mg/m², about 5.5 mg/m² to about 6.5 mg/m², or about 6.0 mg/m^{2.}

The total dose may be 4.5 mg/m². The total dose may be 5.0 mg/m². The total dose may be 7.0 mg/m². The total dose may be 8.0 mg/m^{2.}

The compound may be administered as a single dose during the three-week cycle. The single dose may be about 4.5 mg/m². The single dose may be about 5.0 mg/m^{2.}

The compound may be administered at a dose of 4.5 mg/m² on day 1 of a three-week cycle.

The compound may be administered at a dose of 5.0 mg/m² on day 1 of a three-week cycle.

The compound may be administered as a first dose and a second dose during the three-week cycle. The first dose may be administered on day 1 of the three-week cycle and the second dose may be administered on day 8 of the three-week cycle. The amount of compound administered for the first dose and the amount of compound administered for the second dose may be equal. The total dose for the first dose and the second dose may be about 6.0 mg/m^{2.}

The first dose may be about 3.0 mg/m² and the second dose may be about 3.0 mg/m^{2.}

The total dose for the first dose and the second dose may be about 7.0 mg/m². The first dose may be about 3.5 mg/m² and the second dose may be about 3.5 mg/m^{2.}

The total dose for the first dose and the second dose may be about 8.0 mg/m². The first dose may be about 4.0 mg/m² and the second dose may be about 4.0 mg/m^{2.}

The compound may be administered at a dose of 3.0 mg/m² on day 1, day 8 of a three-week cycle.

The compound may be administered at a dose of 3.5 mg/m² on day 1, day 8 of a three-week cycle.

The compound may be administered at a dose of 4.0 mg/m² on day 1, day 8 of a three-week cycle.

The compound may be administered parentally, preferably intravenously.

The cancer may be selected from lung cancer including non-small cell lung cancer and small cell lung cancer, colon cancer, rectal cancer, colorectal cancer, breast cancer, pancreas cancer, sarcoma including soft tissue sarcoma and bone sarcoma, ovarian cancer, prostate cancer, gastric cancer, renal cancer, melanoma, neuroendocrine tumor, endometrial cancer, adenoid cystic carcinoma, and adrenocortical carcinoma. The renal cancer may be renal carcinoma, kidney clear cell carcinoma or hypernephroma, including poorly differentiated hypernephroma. The melanoma may be amelanotic melanoma. The soft tissue sarcoma may be selected from fibrosarcoma, leiomyosarcoma and liposarcoma. The bone sarcoma may be chondrosarcoma, including myxoid chondrosarcoma.

According to a further aspect of the present invention there is provided a compound of formula 1:

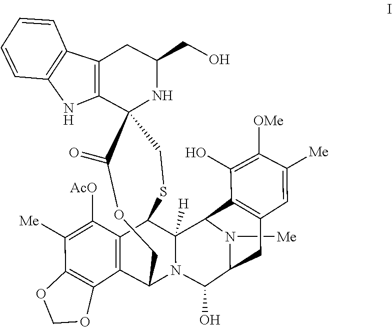 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

or a pharmaceutically acceptable salt or ester thereof, for use in the treatment of a cancer selected from renal cancer, melanoma, neuroendocrine tumor, endometrial cancer, adenoid cystic carcinoma, adrenocortical carcinoma, bone sarcoma and soft tissue sarcoma.

The present invention has provided for the first time data demonstrating the efficacy of the compound of formula I in the cancers disclosed herein.

The cancer may be renal cancer and may be selected from renal carcinoma, kidney clear cell carcinoma and hypernephroma, wherein the hypernephroma may be poorly differentiated hypernephroma.

The cancer may be melanoma and may be amelanotic melanoma.

The cancer may be soft tissue sarcoma and may be selected from leiomyosarcoma and Iiposarcoma.

The cancer may be bone sarcoma and may be chondrosarcoma, including myxoid chondrosarcoma.

The salt may be selected from hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate, p-toluenesulfonate, sodium, potassium, calcium, ammonium, ethylenediamine, ethanolamine, N,N-dialkylenethanolamine, triethanolamine and basic amino acids.

In a further aspect, there is provided a pharmaceutical composition comprising a compound of formula I or a pharmaceutically acceptable salt or ester thereof, and a pharmaceutically acceptable carrier, for use as defined herein.

In a further aspect, there is provided a dosage form comprising a pharmaceutical composition as defined herein, for use as defined herein.

In a further aspect, there is provided a kit comprising a compound, composition or dosage form as defined herein, together with instructions for use as defined herein.

In a further aspect, there is provided a compound of formula I, or a pharmaceutically acceptable salt or ester thereof, or a pharmaceutical composition as defined herein, or a dosage form as defined herein when used according to a use as defined herein.

In a further aspect, there is provided use of a compound of formula I, or a pharmaceutically acceptable salt or ester thereof, or a pharmaceutical composition as defined herein, or a dosage form as defined herein in the manufacture of a medicament for the treatment of a cancer, wherein the compound is administered as defined herein.

In a further aspect, there is provided a method of treating a cancer in a patient in need thereof, comprising administering to the patient a therapeutically effective amount of a compound of formula I, or a pharmaceutically acceptable salt or ester thereof, or a pharmaceutical composition as defined herein, or a dosage form as defined herein, wherein the compound is administered as defined herein.

### BRIEF DESCRIPTION OF THE DRAWINGS

The present invention is further described in the following non-limiting figures.

FIG. 1 shows tumor growth (mean) and body weight (inset) curves for mice (N=10/group) bearing MRI-H-121 xenografts and treated with Placebo or PM14 (PM140014).

FIG. 2 shows tumor growth (median) curves for mice (N=10/group) bearing MRI-H-121 xenografts and treated with Placebo or PM14 (PM140014).

FIG. 3 shows Kaplan-Meier survival curve obtained in mice bearing MRI-H-121 xenografts and treated with Placebo or PM14 (PM140014).

FIG. 4A shows efficacy data in human clinical trials at various doses according to Schedule A (D1,D8).

FIG. 4B shows efficacy data in human clinical trials at various doses according to Schedule B (D1).

FIG. 5A shows dose-limiting toxicity data in human clinical trials at various doses according to Schedule A (D1,D8).

FIG. 5B shows dose-limiting toxicity data in human clinical trials at various doses according to Schedule B (D1).

FIG. 6 shows pharmacokinetic data for: (A) PM14 plasma concentration vs. time; (B) dose adjusted PM14 vs. time.

FIG. 7 shows simulations of PM14 pharmacokinetics with different doses and infusion-rates.

### DETAILED DESCRIPTION OF THE INVENTION

The following apply to all aspects of the present invention.

PM14 is a synthetic compound under clinical investigation. PM14 is a specific inhibitor of oncogenic transcription. PM14 has demonstrated encouraging pre-clinical activity in several cancer models. PM14 was first disclosed in WO2018/197663 (as compound 4-S). The structure for PM14 is:

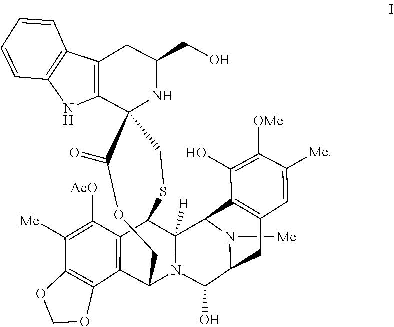 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

PM14 binds DNA forming adducts that specifically inhibit the active transcription of protein-coding genes, by inhibiting mRNA synthesis. The mechanism of transcription inhibition involves the irreversible degradation of elongating RNA polymerase II (Pol II) and the subsequent generation of DNA double strand breaks. As a final effect, PM14 induces the arrest of the cell cycle on the S-phase and apoptosis of the tumor cell.

The terms “pharmaceutically acceptable salt” and “ester” refers to any pharmaceutically acceptable salt or ester which, upon administration to the patient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of the compounds provided herein are synthesized from the parent compounds, which contain a basic or acidic moiety, by conventional chemical methods. Generally, such salts are, for example, prepared by reacting the free acid or base of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts.

The compounds of the invention may be in crystalline or amorphous form either as free compounds or as solvates (e.g. hydrates) and it is intended that all forms are within the scope of the present invention. Methods of solvation are generally known within the art.

In addition, compounds referred to herein may exist in isotopically-labelled forms. All pharmaceutically acceptable salts, esters and isotopically labelled forms of the compounds referred to herein, and mixtures thereof, are considered within the scope of the present invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

In the present application, by “cancer” it is meant to include tumors, neoplasias and any other malignant disease having as cause malignant tissue or cells.

As used herein, the term “treating”, unless otherwise indicated, means reversing, attenuating, alleviating, delaying or inhibiting the progress of the disease or condition to which such term applies, or one or more symptoms of such disorder or condition.

As used herein, the term “subject” refers to a living organism that is treated with a compound of the present invention, including a mammal, such as a human, other primates, sports animals, animals of commercial interest such as cattle, farm animals such as horses, or pets such as dogs and cats. Preferably, the subject is a human.

Soft Tissue Sarcomas

Soft tissue sarcomas can affect any part of the body. They develop in supporting or connective tissue such as the muscle, nerves, fatty tissue, and blood vessels. Soft tissue sarcomas include: GIST which is a common type of sarcoma which develops in the gastrointestinal (GI) tract; gynaecological sarcomas which occur in the female reproductive system: the uterus (womb), ovaries, vagina, vulva and fallopian tubes; and retroperitoneal sarcomas which occur in the retroperitoneum.

Unless detected at an early stage when the tumour can be removed by surgery there is currently no cure for soft tissue sarcoma. Approximately 16% of patients with soft tissue sarcoma have advanced stage (metastatic) disease. For these patients, the relative 5 year survival rate is 16% (American Cancer Society).

Liposarcoma

One particular soft tissue sarcoma is liposarcoma. Liposarcoma is a rare cancer of connective tissues that resemble fat cells under a microscope. It accounts for up to 18% of all soft tissue sarcomas. Liposarcoma can occur in almost any part of the body, but more than half of liposarcoma cases involve the thigh, and up to a third involve the abdominal cavity. Liposarcoma tends to affects adults between the ages of 40 and 60. When it does occur in children, it is usually during the teenage years. There are four types of liposarcoma as shown below. The risk of recurrence and metastasis with liposarcoma increases with higher grade.

Well-differentiated liposarcoma is the most common subtype and usually starts as a low grade tumour. Low grade tumour cells look much like normal fat cells under the microscope and tend to grow and change slowly.

Myxoid liposarcoma is an intermediate to high grade tumour. Its cells look less normal under the microscope and may have a high grade component.

Dedifferentiated liposarcoma occurs when a low grade tumour changes, and the newer cells in the tumour are high grade.

Leiomyosarcoma

Leiomyosarcoma, or LMS, is a type of rare cancer that grows in the smooth muscles. The smooth muscles are in the hollow organs of the body, including the intestines, stomach, bladder, and blood vessels. In females, there is also smooth muscle in the uterus. These smooth muscle tissues help move blood, food, and other material through the body and work without you being aware. LMS is an aggressive cancer and is found most often in the abdomen or in the uterus. LMS is a type of soft tissue sarcoma and makes up between 10% to 20% of soft tissue sarcoma cases. LMS is more common in adults than children. It is estimated that only about 20 to 30 children are diagnosed with LMS in the United States per year. LMS of the uterus affects about 6 per 1 million people per year in the United States. Certain genetic conditions are believed to be associated with LMS, including hereditary retinoblastoma, Li-Fraumeni syndrome, neurofibromatosis type 1, tuberous sclerosis, nevoid basal cell carcinoma syndrome, Gardner syndrome, and Werner syndrome.

Bone Sarcoma

There are various different primary bone cancer, which are named on the part of the bone or nearby tissue that's affected and the kind of cells forming the tumor.

Chondrosarcoma

Chondrosarcoma starts in cartilage cells and is the second most common primary bone cancer. It's rare in people younger than 20. After age 20, the risk of getting a chondrosarcoma goes up until about age 75. Women get this cancer as often as men.

Chondrosarcomas can start anywhere there is cartilage. Most develop in bones like the pelvis, legs, or arms but can also start in the trachea, larynx, chest wall, scapula, ribs, or skull.

Extraskeletal Myxoid Chondrosarcoma

Extraskeletal myxoid chondrosarcoma (EMS) or myxoid chondrosarcoma (and also called EMC) is a rare, slow-growing type of cancer that forms in soft tissues outside the bone, and usually has certain changes in the NR4A3 gene that result in special fusion proteins to be made. Extraskeletal myxoid chondrosarcomas usually occur in the thigh, but may also occur in the knee, buttock, or trunk (chest and abdomen). They may grow large and spread to nearby tissue or to other parts of the body, especially the lungs. They may also recur many years after treatment. Extraskeletal myxoid chondrosarcomas usually occur in middle-aged or older adults and are rare in children and adolescents.

Fibrosarcoma

Fibrosarcoma develops more often in soft tissues than it does in bones. Fibrosarcoma usually occurs in elderly and middle-aged adults. Bones in the legs, arms, and jaw are most often affected.

Melanoma

Melanoma is a type of skin cancer that develops when melanocytes (the cells that give the skin its tan or brown color) start to grow out of control. Melanoma is much less common than some other types of skin cancers. But melanoma is more dangerous because it's much more likely to spread to other parts of the body if not caught and treated early. Around 16,200 people are diagnosed with melanoma in the UK each year. The number of people diagnosed with melanoma has increased over the last few decades. Melanoma is the 5th most common cancer in the UK.

Melanomas can develop anywhere on the skin, but they are more likely to start on the trunk (chest and back) in men and on the legs in women. The neck and face are other common sites.

Amelanotic melanoma is a form of melanoma in which the malignant cells have little to no pigment. The term ‘amelanotic’ is often used to indicate lesions that are only partially devoid of pigment while truly amelanotic melanoma where lesions lack all pigment is rare.

Having darkly pigmented skin lowers your risk of melanoma at these more common sites, but anyone can get melanoma on the palms of the hands, soles of the feet, or under the nails. Melanomas in these areas make up a much larger portion of melanomas in African Americans than in whites.

Melanomas can also form in other parts of your body, such as the eyes, mouth, genitals, and anal area, but these are much less common than melanoma of the skin.

Neuroendocrine Tumor

Pancreatic neuroendocrine tumors (NETs), or islet cell tumors, are a type of cancer that starts in the pancreas. Pancreatic NETs are a less common type of pancreatic cancer. They make up less than 2% of pancreatic cancers, but tend to have a better prognosis than the more common type. Pancreatic neuroendocrine tumors start in neuroendocrine cells. Although neuroendocrine cells (or endocrine cells) are also found in other areas of the body, only cancers that form from neuroendocrine cells in the pancreas are called pancreatic neuroendocrine tumors.

Neuroendocrine cells in the pancreas are found in small clusters called islets (or islets of Langerhans). These islets make hormones like insulin and glucagon and release them directly into the blood.
Grade 1 (also called low-grade or well-differentiated) neuroendocrine tumors have cells that look more like normal cells and are not multiplying quickly.
Grade 2 (also called intermediate-grade or moderately differentiated) tumors have features in between those of low- and high-grade tumors.
Grade 3 (also called high-grade or poorly differentiated) neuroendocrine tumors have cells that look very abnormal and are multiplying faster. These are also know as neuroendocrine carcinomas (NECs).

Pancreatic NETs are also named based on whether they are or non-functioning.

Functioning NETs make hormones that are released into the blood and cause symptoms.

Most (up to 70%) functioning NETs are insulinomas. The other types are much less common:
Insulinomas come from cells that make insulin.
Glucagonomas come from cells that make glucagon.
Gastrinomas come from cells that make gastrin.
Somatostatinomas come from cells that make somatostatin.
VlPomas come from cells that make vasoactive intestinal peptide (VIP).
ACTH-secreting tumors come from cells that make adrenocorticotropic hormone (ACTH).

Non-functioning NETs don't make enough excess hormones to cause symptoms and can therefore often grow quite large before they're found. Symptoms that may occur when they grow to a large size include abdominal (belly) pain, lack of appetite, and weight loss.

Carcinoid tumors are much more common in other parts of the digestive system, although rarely they can start in the pancreas. These tumors often make serotonin.

Endometrial Cancer

Endometrial cancer (also called endometrial carcinoma) starts in the cells of the inner lining of the uterus (the endometrium). This is the most common type of cancer in the uterus.

Endometrial carcinomas can be divided into different histologic types including:
Adenocarcinoma
Uterine carcinosarcoma or CS
Squamous cell carcinoma
Small cell carcinoma
Transitional carcinoma
Serous carcinoma

Clear-cell carcinoma, mucinous adenocarcinoma, undifferentiated carcinoma, dedifferentiated carcinoma, and serous adenocarcinoma are less common types of endometrial adenocarcinomas. They tend to grow and spread faster than most types of endometrial cancer. They often have spread outside the uterus by the time they're diagnosed.

Endometrioid cancer—most endometrial cancers are adenocarcinomas, and endometrioid cancer is by far the most common type of adenocarcinoma. Endometrioid cancers start in gland cells and look a lot like the normal uterine lining (endometrium). Some of these cancers have squamous cells (squamous cells are flat, thin cells), as well as glandular cells. There are many endometrioid cancer sub-types including:
Adenocarcinoma, (with squamous differentiation)
Adenoacanthoma
Adenosquamous (or mixed cell)
Secretory carcinoma
Ciliated carcinoma
Villoglandular adenocarcinoma

Uterine carcinosarcoma (CS) starts in the endometrium and has features of both endometrial carcinoma and sarcoma. In the past, CS was considered a different type of uterine cancer called uterine sarcoma but it is no believed to be a poorly differentiated endometrial carcinoma.

Uterine CS is a type 2 endometrial carcinoma. CS tumors are also known as malignant mixed mesodermal tumors or malignant mixed mullerian tumors (MMMTs). They make up about 3% of uterine cancers.

Adenoid Cystic Carcinoma

Adenoid cystic carcinoma (ACC) is a rare form of adenocarcinoma, a type of cancer that begins in glandular tissues. It most commonly arises in the major and minor salivary glands of the head and neck. It can also occur in the breast, uterus, or other locations in the body.

Adrenocortical Carcinoma

Adrenal cancer is a rare cancer that begins in one or both of the small, triangular glands (adrenal glands) located on top of the kidneys. Adrenal cancer, also called adrenocortical cancer, can occur at any age. But it's most likely to affect children younger than 5 and adults in their 40s and 50s.

Renal Cancer

Renal (or kidney) cancer is a type of cancer that starts in the kidney. There are a number of types of renal cancer.

Renal cell carcinoma (RCC), also known as renal cell cancer or renal cell adenocarcinoma, is the most common type of kidney cancer. About 9 out of 10 kidney cancers are renal cell carcinomas. Although RCC usually grows as a single tumor within a kidney, sometimes there are 2 or more tumors in one kidney or even tumors in both kidneys at the same time. There are several histologic subtypes of RCC:
Clear cell renal cell carcinoma: this is the most common form of renal cell carcinoma. About 7 out of 10 people with RCC have this kind of cancer. The cells that make up clear cell RCC look very pale or clear.
Non-clear cell renal cell carcinomas:
   Papillary renal cell carcinoma (also called chromophilic): This is the second most common subtype—about 1 in 10 RCCs are of this type. These cancers form papillae in some, if not most, of the tumor.
   Chromophobe renal cell carcinoma: This subtype accounts for about 5% (5 cases in 100) of RCCs. The cells of these cancers are also pale, like the clear cells, but are much larger and have certain other features that can be recognized when looked at very closely.
Rare types of renal cell carcinoma: These subtypes are very rare, each making up less than 1% of RCCs:
   Collecting duct RCC
   Multilocular cystic RCC
   Medullary carcinoma
   Mucinous tubular and spindle cell carcinoma
   Neuroblastoma-associated RCC
Unclassified renal cell carcinoma: Rarely, renal cell cancers are labeled as unclassified because the way they look doesn't fit into any of the other categories or because there is more than one type of cancer cell present.

Other types of kidney cancers include:
Transitional cell carcinoma: Of every 100 cancers in the kidney, about 5 to 10 are transitional cell carcinomas (TCCs), also known as urothelial carcinomas.
Wilms tumor (nephroblastoma): Wilms tumors almost always occur in children. This type of cancer is very rare among adults.
Renal sarcoma: Renal sarcomas are a rare type of kidney cancer that begin in the blood vessels or connective tissue of the kidney. They make up less than 1% of all kidney cancers.

According to an embodiment of the present invention, there is provided a compound of formula I:

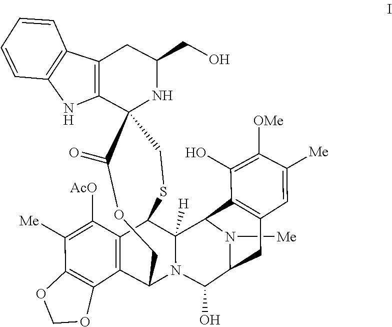 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

or a pharmaceutically acceptable salt or ester thereof, for use in the treatment of a cancer selected from renal cancer, melanoma, neuroendocrine tumor, endometrial cancer, adenoid cystic carcinoma, adrenocortical carcinoma, bone sarcoma and soft tissue sarcoma.

In a preferred embodiment, the renal cancer is renal carcinoma, kidney clear cell carcinoma or hypernephroma. The hypernephroma may be poorly differentiated hypernephroma.

In a preferred embodiment, the melanoma is amelanotic melanoma.

In a preferred embodiment, the soft tissue sarcoma is selected from leiomyosarcoma and liposarcoma. In a preferred embodiment, the soft tissue sarcoma excludes fibrosarcoma.

In a preferred embodiment, the bone sarcoma is chondrosarcoma, including myxoid chondrosarcoma.

According to another embodiment of the present invention, there is provided a compound of formula I:

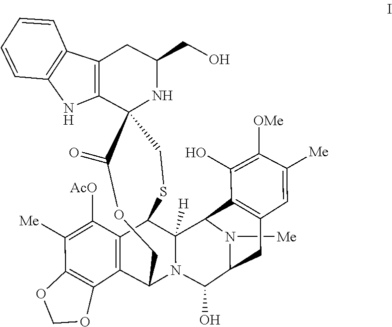 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]

or a pharmaceutically acceptable salt or ester thereof, for use in the treatment of cancer, wherein the compound is administered to a subject in a three-week cycle at a total dose of from about 0.5 mg/m² to about 9 mg/m^{2.}

As used herein, the term “total dose” refers to the total amount of compound administered during the three-week cycle.

In a preferred embodiment, the total dose is about 1.0 mg/m² to about 9.0 mg/m², about 1.5 mg/m² to about 9.0 mg/m², about 2.0 mg/m² to about 9.0 mg/m², about 2.5 mg/m² to about 8.5 mg/m², about 3.0 mg/m² to about 8.0 mg/m², about 3.5 mg/m² to about 7.5 mg/m², about 4.0 mg/m² to about 7.0 mg/m², about 4.0 mg/m² to about 6.5 mg/m², about 4.5 mg/m² to about 6.5 mg/m², about 4.5 mg/m² to about 6.0 mg/m^{2.}

In a preferred embodiment, the total dose is about 3.0 mg/m² to about 6.0 mg/m², about 3.0 mg/m² to about 5.6 mg/m², about 3.5 mg/m² to about 5.6 mg/m², about 4.0 mg/m² to about 5.0 mg/m² or about 4.5 mg/m^{2.}

In a preferred embodiment, the total dose is about 4.0 mg/m² to about 9.0 mg/m², about 4.0 mg/m² to about 8.0 mg/m², about 4.5 mg/m² to about 7.5 mg/m², about 5.0 mg/m² to about 7.0 mg/m², about 5.5 mg/m² to about 6.5 mg/m², or about 6.0 mg/m^{2.}

In a preferred embodiment, the total dose is about 4.5 mg/m² to about 8.0 mg/m², about 4.5 mg/m² to about 5.0 mg/m², about 7.0 mg/m² to about 8.0 mg/m², about 4.5 mg/m², about 5.0 mg/m², about 7.0 mg/m², or about 8.0 mg/m^{2.}

The compound may be administered in one or more doses during the three-week cycle. For example, the compound may be administered 1, 2, 3, 4, 5, 6, 7, 8, 9 or 10 times during the three-week cycle. In some embodiments, the compound may be administered once weekly. In other embodiments, the compound may be administered once daily.

The total dose may be split evenly between each individual dose within the three-week cycle. Said another way, the amount of compound administered for each dose may be equal.

In some embodiments, the compound of formula I is administered as a single dose during the three-week cycle.

Preferably, the single dose is about 3.0 mg/m² to about 6.0 mg/m², more preferably about 3.0 mg/m² to about 5.6 mg/m², more preferably about 3.5 mg/m² to about 5.6 mg/m², even more preferably about 4.0 mg/m² to about 5.0 mg/m². It is particularly preferred that the single dose is about 4.5 mg/m². The single dose may be administered on day 1 of the cycle.

It is further particularly preferred that the single dose is about 5.0 mg/m². The single dose may be administered on day 1 of the cycle.

In a particularly preferred embodiment, the compound of formula I is administered at a dose of 4.5 mg/m² on day 1 of a three-week cycle.

In a further particularly preferred embodiment, the compound of formula I is administered at a dose of 5.0 mg/m² on day 1 of a three-week cycle.

In some embodiments, the compound of formula I is administered 2, 3, 4, 5, 6, 7, 8, 9 or 10 times during the cycle. In some embodiments the compound is administered 3 times during the cycle. In some embodiments the compound is administered 3 times during the three-week cycle.

In some embodiments the compound is administered 3 times during the cycle on days 1, 2 and 3.

Preferably, the amount of compound administered for each of the doses may be equal. For example, if the drug is administered three times on days 1, 2 and 3 of a three-week cycle, the dose administered on each of these days is the same.

Preferably, the total dose is about 0.5 mg/m², 1.0 mg/m², 1.5 mg/m², 2.0 mg/m², 2.5 mg/m^{2, 3.0} mg/m², 3.5 mg/m², 4.0 mg/m², 4.5 mg/m^{2, 5.0} mg/m², 5.5 mg/m^{2, 6.0} mg/m², 6.5 mg/m^{2, 7.0} mg/m², 7.5 mg/m², 8.0 mg/m², 8.5 mg/m², 9.0 mg/m², 9.5 mg/m², 10.0 mg/m², 10.5 mg/m2 or 11.0 mg/m^{2.}

Preferably, each individual dose (i.e. each day) is about 0.5 mg/m², 1.0 mg/m², 1.5 mg/m², 2.0 mg/m², 2.5 mg/m², 3.0 mg/m², 3.5 mg/m², 4.0 mg/m², 4.5 mg/m², 5.0 mg/m², 5.5 mg/m², 6.0 mg/m², 6.5 mg/m², 7.0 mg/m², 7.5 mg/m², 8.0 mg/m², 8.5 mg/m² or 9.0 mg/m^{2.}

In some embodiments, the compound of formula I is administered as a first dose and a second dose during the three-week cycle (i.e. 2 times during the three-week schedule). Preferably, the first dose is administered on day 1 of the three-week cycle and the second dose is administered on day 8 of the three-week cycle.

Preferably, the amount of compound administered for the first dose and the amount of compound administered for the second dose are equal.

Preferably, the total dose for the first dose and the second dose is about 0.5 mg/m² to about 9.0 mg/m², more preferably about 1.0 mg/m² to about 9.0 mg/m², more preferably about 1.5 mg/m² to about 9.0 mg/m², more preferably about 2.0 mg/m² to about 9.0 mg/m², more preferably about 3.0 mg/m² to about 9.0 mg/m², more preferably about 4.0 mg/m² to about 9.0 mg/m², more preferably about 5.0 mg/m² to about 9.0 mg/m², more preferably about 6.0 mg/m2 to about 9.0 mg/m², more preferably about 4.0 mg/m² to about 8.0 mg/m², more preferably about 6.0 mg/m² to about 9.0 mg/m², more preferably about 5.0 mg/m² to about 7.0 mg/m2, even more preferably about 5.5 mg/m² to about 6.5 mg/m². It is particularly preferred that the total dose for the first dose and the second dose is about 6.0 mg/m^{2.}

Preferably, the first dose and/or the second dose is about 2.25 mg/m² to about 3.75 mg/m2, more preferably about 2.5 mg/m² to about 3.5 mg/m², even more preferably about 2.75 mg/m2 to about 3.25 mg/m². It is particularly preferred that the first dose and/or the second dose is about 3.0 mg/m^{2.}

In a particularly preferred embodiment, the total dose for the first dose and the second dose is about 6.0 mg/m² to about 9.0 mg/m², more preferably about 6.5 mg/m² to about 8.5 mg/m2, more preferably about 7.0 mg/m² to about 8.0 mg/m², more preferably 7.0 mg/m² or 8.0 mg/m^{2.}

Preferably, the first dose and/or the second dose is about 3.0 mg/m² to about 4.5 mg/m², more preferably about 3.25 mg/m² to about 4.25 mg/m², even more preferably about 3.5 mg/m² to about 4.0 mg/m². It is particularly preferred that the first dose and/or the second dose is about 3.5 mg/m2 or about 4.0 mg/m^{2.}

In a particularly preferred embodiment, the compound of formula I is administered at a dose of 3.0 mg/m² on day 1 and at a dose of 3.0 mg/m² on day 8 of a three-week.

In a further particularly preferred embodiment, the compound of formula I is administered at a dose of 3.5 mg/m² on day 1 and at a dose of 3.5 mg/m² on day 8 of a three-week.

In a further particularly preferred embodiment, the compound of formula I is administered at a dose of 4.0 mg/m² on day 1 and at a dose of 4.0 mg/m² on day 8 of a three-week.

In some embodiments, the compound is administered parentally. Preferably, the compound is administered intravenously.

The dosage regimens as disclosed herein are useful in the treatment of cancer. In a preferred embodiment, the cancer is selected from lung cancer including non-small cell lung cancer and small cell lung cancer, colon cancer, rectal cancer, colorectal cancer, breast cancer, pancreas cancer, sarcoma including soft tissue sarcoma or bone sarcoma, ovarian cancer, prostate cancer, gastric cancer, renal cancer, melanoma, neuroendocrine tumor, endometrial cancer, adenoid cystic carcinoma and adrenocortical carcinoma.

In a preferred embodiment, the lung cancer is non-small cell lung cancer or small cell lung cancer.

In a preferred embodiment, the renal cancer is renal carcinoma, kidney clear cell carcinoma or hypernephroma. The hypernephroma may be poorly differentiated hypernephroma.

In a preferred embodiment, the melanoma is amelanotic melanoma.

In a preferred embodiment, the sarcoma is soft tissue sarcoma.

In a preferred embodiment, the soft tissue sarcoma is selected from fibrosarcoma, leiomyosarcoma and liposarcoma.

In a preferred embodiment, the sarcoma is bone sarcoma.

In a preferred embodiment, the bone sarcoma is chondrosarcoma, including myxoid chondrosarcoma.

In a further embodiment of the present invention, there is provided a pharmaceutical composition comprising a compound of formula I or a pharmaceutically acceptable salt or ester thereof, and a pharmaceutically acceptable carrier, for use in the treatment of a cancer as described herein.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example inhalatory administration. Powders may also be used for inhalation dosage forms. The term “carrier” refers to a diluent, adjuvant or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions according to the present invention, and pharmaceutically acceptable carriers are sterile. Water is a preferred carrier when the compounds according to the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

Examples of the administration form include without limitation oral, topical, parenteral, sublingual, rectal, vaginal, ocular and intranasal. Parenteral administration includes subcutaneous injections, intravenous, intramuscular, intrasternal injection or infusion techniques. Preferably the compositions are administered parenterally.

Pharmaceutical compositions of the invention can be formulated so as to allow a compound according to the present invention to be bioavailable upon administration of the composition to an animal, preferably human. Compositions can take the form of one or more dosage units, where for example, a tablet can be a single dosage unit, and a container of a compound according to the present invention may contain the compound in liquid or in aerosol form and may hold a single or a plurality of dosage units.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more for the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agent such as sucrose or saccharin; a flavoring agent such as peppermint, methyl salicylate or orange flavoring; and a coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, the compounds according to the present invention are administered intravenously. Infusion times of up to 24 hours are preferred to be used, more preferably 1 to 12 hours, with 1 to 6 hours being most preferred. An infusion time may be 24 hours. Further infusion times include 1, 2, 3, 4, 5 or 6 hours. An infusion time may be three hours. Short infusion times which allow treatment to be carried out without an overnight stay in a hospital are especially desirable.

However, infusion may be 12 to 24 hours or even longer if required. Infusion may be carried out at suitable intervals of, for example, 1 to 4 weeks.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides, polyethylene glycols, glycerin, or other solvents; antibacterial agents such as benzyl alcohol or methyl paraben; and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant.

Typically, the amount is at least about 0.01% of a compound of the present invention, and may comprise at least 80%, by weight of the composition. When intended for oral administration, this amount can be varied to range from about 0.1% to about 80% by weight of the composition. Preferred oral compositions can comprise from about 4% to about 50% of the compound of the present invention by weight of the composition.

Preferred compositions of the present invention are prepared so that a parenteral dosage unit contains from about 0.01% to about 10% by weight of the compound of the present invention. More preferred parenteral dosage unit contains about 0.5% to about 5% by weight of the compound of the present invention.

The compound of the present invention, can be administered by any convenient route, for example by infusion or bolus injection, by absorption through epithelial or mucocutaneous linings.

In specific embodiments, it can be desirable to administer one or more compounds of the present invention, or compositions locally to the area in need of treatment. In one embodiment, administration can be by direct injection at the site (or former site) of a cancer, tumor or neoplastic or pre-neoplastic tissue.

Pulmonary administration can also be employed, e.g. by use of an inhaler or nebulizer, and formulation with an aerosolizing agent, or via perfusion in a fluorocarbon or synthetic pulmonary surfactant. In certain embodiments, the compound of the present invention can be formulated as a suppository, with traditional binders and carriers such as triglycerides.

The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin.

The pharmaceutical compositions can be prepared using methodology well known in the pharmaceutical art. For example, a composition intended to be administered by injection can be prepared by combining a compound of the present invention with water, or other physiologically suitable diluent, such as phosphate buffered saline, so as to form a solution. A surfactant can be added to facilitate the formation of a homogeneous solution or suspension.

Preferred compositions according to the present invention include:
Pharmaceutical compositions comprising a compound of the present invention and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.
Lyophilised pharmaceutical compositions comprising a compound of the present invention and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.

The ratio of the active substance to the disaccharide in embodiments of the present invention is determined according to the solubility of the disaccharide and, when the formulation is freeze dried, also according to the freeze-dryability of the disaccharide. It is envisaged that this active substance:disaccharide ratio (w/w) can be about 1:10 in some embodiments, about 1:20 in other embodiments, about 1:50 in still other embodiments. It is envisaged that other embodiments have such ratios in the range from about 1:5 to about 1:500, and still further embodiments have such ratios in the range from about 1:10 to about 1:500.

The composition comprising a compound of the present invention may be lyophilized. The composition comprising a compound of the present invention is usually presented in a vial which contains a specified amount of such compound.

The compound according to the present invention can be administered to an animal that has also undergone surgery as treatment for the cancer. In one embodiment of the present invention, the additional method of treatment is radiation therapy. In a specific embodiment of the present invention, the compound according to the present invention is administered concurrently with radiation therapy. In another specific embodiment, the radiation therapy is administered prior or subsequent to administration of the compound of the present invention, preferably at least an hour, three hours, five hours, 12 hours, a day, a week, a month, more preferably several months (e.g. up to three months) prior or subsequent to administration of a compound or composition of the present invention.

Any radiation therapy protocol can be used depending upon the type of cancer to be treated. For example, but not by way of limitation, x-ray radiation can be administered; in particular, high-energy megavoltage (radiation of greater than 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

It has found that the compounds of the present invention and compositions of the present invention are particularly effective in the treatment of certain types of cancer.

Thus, the compounds and compositions according to the present invention are useful for inhibiting the multiplication, or proliferation, of a tumor cell or cancer cell, or for treating cancer in an animal, preferably a human.

The present invention is further described in the following non-limiting examples.

### Examples

PM14 can be obtained following the teaching of WO2018/197663, the contents of which are herein incorporated by reference.

### Example 1: Renal cancer activity in RXF 393 and Caki-1 in vitro assays

Cell lines and cell culture: The following human cancer cell lines have been used in this example (the collection code and the tissue of origin are shown in brackets):
RXF 393 (NCI) (renal carcinoma)
Caki-1 (ATCC HTB-46) (kidney clear cell carcinoma)

Cell lines were obtained from the American Type Culture Collection (ATCC) or the National Cancer Institute (NCI). Cells were maintained in the appropriate culture medium, namely:
RPMI for RXF 393
McCoy's 5A for Caki-1

All media were supplemented with 10% FBS, 2 mM L-Glutamine and 100 units/mL of penicillin-streptomycin.

Cell viability assay: To assess the antiproliferative activity of the compounds a colorimetric assay based on 3-(4,5-Dimethylthiazol-2-yl)-2,5-diphenyltetrazolium bromide (MTT) reduction was used. MTT is a tetrazolium salt which is reduced to a purple formazan by functional mitochondria, hence purple colour intensity is proportional to the amount of living cells. An appropriate number of cells, to reach a final cell density in the assay ranging from 5,000 to 15,000 cells per well depending on the cell line, were seeded in 96-well plates and allowed to stand in culture medium for 24 h at 37° C. under 5% CO₂ and 98% humidity. Then, compounds or DMSO in culture medium were added to reach a final volume of 200 pL and the intended compound concentration in a range covering 10 serial 2/5 dilutions starting from 0.1 pg/mL (10 μg/mL for doxorubicin) in 1% (v/v) DMSO. At this point a set of “time zero control plates” treated with 1% (v/v) DMSO were processed with MTT as described below. The rest of the plates were incubated during 72 h under the aforementioned environmental conditions. Afterwards 50 μL of a 1 mg/mL MTT solution in culture medium were added to the wells and incubated for 6-8 hours at 37° C. to allow formazan crystals generation. Culture medium was then removed and 100 μL of neat DMSO added to each well to dissolve the formazan product into a coloured solution whose absorbance at 540 nm was finally measured in a PolarStar Omega microplate multilabel reader (BMG Labtech, Ortenberg, Germany).

All evaluations were performed in triplicate and the resulting data were fitted by nonlinear regression to a four-parameters logistic curve with Prism v5.0 software (GraphPad Software, La Jolla, CA, USA) following the algorithm developed by the American National Cancer Institute (NCI) (Boyd MR and Paull KD (1995) Drug Dev. Res. 34: 91-104). Such algorithm allow the calculation of three parameters defining the compound effect: GI₅₀ (compound concentration that produces 50% of cell growth inhibition as compared to control cultures), TGI (compound concentration that causes total cell growth inhibition, i.e. a cytostatic effect, as compared to control cultures) and LC₅₀ (compound concentration that produces 50% of net cell killing cytotoxic effect). Briefly, if “Tz” is the number of cells at time zero, “C” is the number of cells after 72 h in DMSO-treated control wells, and “T” is the number of cells after 72 h in test wells, two different scenarios can be considered:
1. If Tz<T<C (i.e. no effect or growth inhibition), cell survival is defined as:

$$\\ \% cell survival = 100 \cdot \frac{T - {T}_{z}}{C - {T}_{z}}$$

2. If T<Tz (i.e. net cell killing), cell survival is defined as:

$$\\ \% cell survival = 100 \cdot \frac{T - {T}_{z}}{{T}_{z}}$$

GI₅₀ is finally used as reference value. Results presented here correspond to the geometric mean of the GI₅₀s obtained in at least three independent experiments each of them was performed in triplicate for every compound in every tumour cell line. To define a significant (ca. 70%) confidence interval for the geometric mean, its value must be multiplied and divided by the corresponding geometric standard deviation (GSD) which was also calculated. When the GSD was greater than 4, outliers were identified among the replicates and the mean GI50 was recalculated ignoring those values to prevent from artifactual bias.

Results: The GI₅₀ values for RXF 393 and Caki-1 are shown below in Table 1, whilst the GSD values are shown below in Table 2.

TABLE 1
GI₅₀ values for RXF 393 and Caki-1

| | GI₅₀, M (geometric |
|---|---|
| | mean, N = 3) |
| Cell line | PM14 |
| RXF 393 | 6.21E−10 |
| Caki-1 | 6.11E−10 |

TABLE 2
GSD values for RXF 393 and Caki-1

| | GSD (N = 3) |
|---|---|
| Cell line | PM14 |
| RXF 393 | 1.36 |
| Caki-1 | 2.07 |

### Example 2: Renal Cancer Activity in RXF 486L and RXF 1781 L In Vitro Assays

Compound handling: PM14 (Pharma Mar) was supplied as powder, shipped frozen at −80° C. and stored at −20° C.

Working stock solution of PM14 was prepared in DMSO at a concentration of 1.042 mM and small aliquots were stored at −20° C. On each day of an experiment, a frozen aliquot of the working stock solution was thawed and stored at room temperature prior to and during treatment.

Subsequent dilutions were done with complete RPMI 1640 cell culture medium. DMSO stock solutions were first diluted 1:22 (corresponding to 4.5% v/v DMSO). Starting with this solution, serial dilutions in half-log steps with cell culture medium were done using an intermediate dilution plate. Finally, 10 μl taken from the intermediate dilution plate were transferred to 140 μl/well of the cell culture plate. Thus, at the highest test concentration the DMSO stock was diluted 1:330, corresponding to a maximum DMSO concentration of 0.3% v/v in the assay.

Cell lines and cell culture: Non-PDX-derived cell lines were either provided by the NCI (Bethesda, MD), or were purchased from ATCC (Rockville, MD) or DSMZ (Braunschweig, Germany). The following human cancer cell lines have been used in this example:
RXF 486L (hypernephroma, poorly differentiated)
RXF 1781L (hypernephroma, poorly differentiated)

Cell lines were routinely passaged once or twice weekly and maintained in culture for up to 20 passages. All cells were grown at 37° C. in a humidified atmosphere with 5% CO₂ in RPMI 1640 medium (25 mM HEPES, with L-glutamine, #FG1385, Biochrom, Berlin, Germany) supplemented with 10% (v/v) fetal calf serum (Sigma, Taufkirchen, Germany) and 0.1 mg/mL gentamicin (Life Technologies, Karlsruhe, Germany).

Cell proliferation assay: A modified propidium iodide (PI) based monolayer assay was used to assess the anti-cancer activity of the compounds (Dengler WA, Schulte J, Berger DP, Mertelsmann R, Fiebig HH, Anti-Cancer Drugs 1995, 6: 522-532). Briefly, cells were harvested from exponential phase cultures, counted and plated in 96 well flat-bottom microtiter plates at a cell density of 4,000 to 30,000 cells/well dependent on the cell line's growth rate. After a 24 h recovery period, to allow the cells to resume exponential growth, 10 μl of culture medium (4 control wells/cell line/plate) or of culture medium with test compounds were added. PM14 was applied at ten concentrations in half-log increments in duplicates up to 3.16 pM and treatment continued for four days. After four days of treatment, cells were next washed with 200 μl PBS to remove dead cells and debris, then 200 μl of a solution containing 7 μg/ml propidium iodide (PI) and 0.1% (v/v) Triton X-100 was added. After an incubation period of 1-2 hours at room temperature, fluorescence (FU) was measured using the Enspire Multimode Plate Reader (excitation A=530 nm, emission A=620 nm) to quantify the amount of attached viable cells.

IC₅₀ and IC₇₀ values were calculated by 4 parameter non-linear curve fit using Oncotest Warehouse Software. For calculation of mean IC50 values, the geometric mean was used.

Results: The IC₅₀ values for RXF 486L and RXF 1781L are shown below in Table 3, whilst the IC₇₀ values for RXF 486L and RXF 1781L are shown below in Table 4.

TABLE 3
IC₅₀ values for RXF 486L and RXF 1781L

| | IC₅₀, μM (geometric mean) |
|---|---|
| Cell line | PM14 |
| RXF 486L | 0.002 |
| RXF 1781L | 0.004 |

TABLE 4
IC₇₀ values for RXF 486L and RXF 1781L

| | IC₇₀, μM (geometric mean) |
|---|---|
| Cell line | PM14 |
| RXF 486L | 0.002 |
| RXF 1781L | 0.005 |

### Example 3: Renal Cancer Activity in MRI-H-121 Mouse Xenograft Studies

Compounds: Vials of off-white lyophilized PM14 cake were stored at −20° C. The cake was reconstituted with 2 ml of water for injection (Sigma-Aldrich, Co) to a concentration of 0.5 mg/ml. Further dilutions were made with a 5% glucose solution for injection/USP (Baxter, Inc.). A clear PM14 solution was obtained.

Placebo: Vials of white to off-white lyophilized placebo cake (composition: sucrose 200 mg, lactic acid 5.52 mg, sodium hydroxide 1.28 mg) were stored at 5° C. The cake was reconstituted with 1.5 ml of water for injection (Sigma-Aldrich, Co). Further dilutions were made with a 5% glucose solution for injection/USP (Baxter, Inc.), resulting in a clear solution.

Animals: Female athymic nu/nu mice between 4 to 6 weeks of age were purchased from Envigo (Barcelona, Spain).

Animals were housed in individually ventilated cages (Sealsafe® Plus, Techniplast S.P.A.): 10 mice per cage, on a 12-hour light-dark cycle at 21-23° C. and 40-60% humidity. Mice were allowed free access to irradiated standard rodent diet (Tecklad 2914C) and sterilized water. Animals were acclimated for five days prior to being individually tattoo-identified.

Animal protocols were reviewed and approved according to regional Institutional Animal Care and Use Committees.

Tumor line: MRI-H-121 is a human renal carcinoma tumor line, originally obtained from the DCT Tumor Bank. Developed by Dr. A. E. Bogden, Mason Research Institute MA and maintained as a serial transplanted tumor line in athymic nude mice. Original tissue came from a patient at U. of Mass. Med. Center.

Study groups: Briefly, 4 to 6 week-old female athymic nu/nu mice were subcutaneously implanted into their right flank with a MRI-H-121 tissue from serial transplanted donor mice. Tumors were removed from donor animals and were cut into fragments (3 mm³). Tissue was debrided of membrane, hemorrhagic and necrotic areas, placed in Matrigel™ (Corning Incorporated Life Sciences) and subcutaneously implanted. Recipient mice were anesthetised by inhalation of isoflurane, a small incision into the skin of the back was made and one tumor fragment per mouse was transplanted with tweezers. The mice were monitored daily.

Tumor bearing animals were randomly allocated into 2 groups (N=10/group): PM14 administered at 1.25 mg/kg and placebo. All treatments were intravenously administered once a week for 3 consecutive weeks (days 0, 7 and 14).

Tumor measurements were determined using digital calipers (Fowler Sylvac, S235PAT). The formula to calculate volume for a prolate ellipsoid was used to estimate tumor volume (mm³) from 2-dimensional tumor measurements:

$$\\ Tumor volume ( {mm}^{3} ) = ( a \cdot {b}^{{}_{} 2} ) / 2.$$

Where, a: length (longest diameter) and b: width (shortest diameter) in mm of a tumor.

Tumor volume and animal body weights were measured 2-3 times per week starting from the first day of treatment.

Treatment tolerability was assessed by monitoring body weight evolution, clinical signs of systemic toxicity, as well as evidences of local damage in the injection site.

Treatments producing >20% lethality and/or 20% net body weight loss were considered toxic. Animals were euthanized when their tumors reached ca. 2,000 mm³ and/or severe necrosis was seen.

When tumors reached ca. 190 mm³, tumor bearing animals were randomly allocated into the following experimental groups (N=10/group):

1. Placebo
2. PM14 (1.25 mg/kg)

Treatments were initiated on Day 0 and were intravenously administered once per week for 3 consecutive weeks (days 0, 7 and 14).

Tumor volume data from groups following the 1st, 2nd, 3rd, 4th and 5th weeks were compared using a two tailed Mann Whitney U test. The data are presented as medians and interquartile range (IQR).

Complete tumor regression (CR) was defined when tumor volume <63 mm³ for 2 or more consecutive measurements. Survival statistical differences between groups were assessed by Kaplan Meier curves applying the log rank test.

Statistical analysis and graphs were performed using GraphPad Prism, version 5.02 (GraphPad Software Inc., San Diego, USA) and NewLab Oncology Software (version 2.25.06.00).

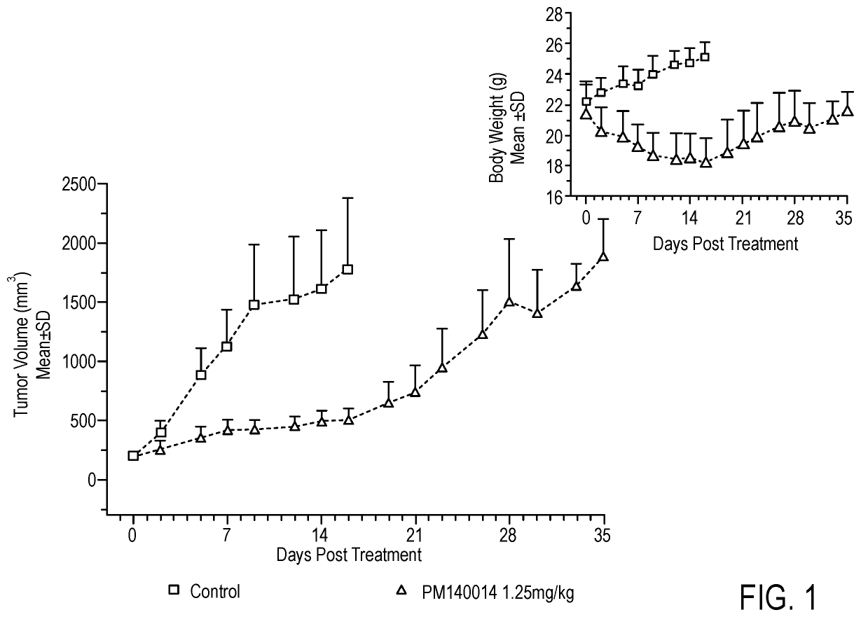

Results: No mortality was registered. PM14 was well tolerated by MRI-H-121 tumor bearing animals, with an important but reversible mean body weight reduction of (ca. −15.0%) recorded on Day 16 (FIG. 1). No other clinical signs of systemic toxicity were seen.

Treatments were initiated on Day 0 when tumors reached a volume of ca. 190 mm³.

Animals in the placebo group were sacrificed due to tumor volume (>2,000 mm³) and/or tumor necrosis between days 9 and 30. In this experiment, MRI-H-121 tumors had a doubling time of 3.2 days.

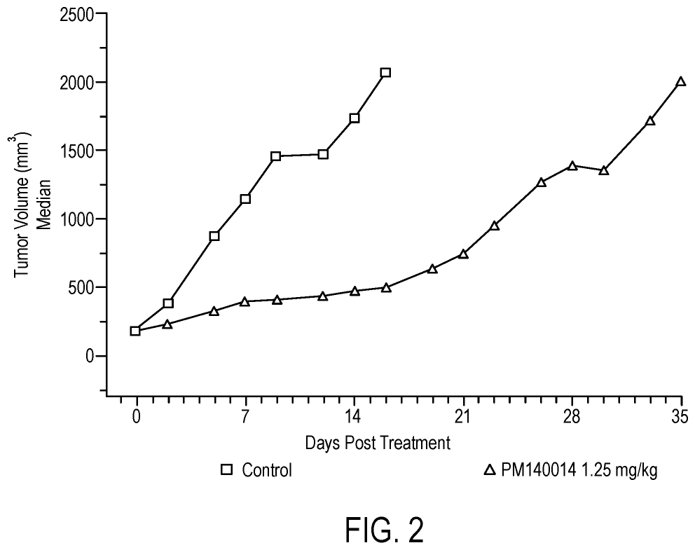

Tumor growth curves are displayed in FIG. 1 and FIG. 2. PM14 showed a very strong antitumor activity in MRI-H-121 tumor xenografts. The placebo-treated group had a median (IQR) tumor volume of 1147 (956.4 to 1468) and 1727 (1228 to 1955) mm³ on days 7 and 14, respectively. On days 7, 14, 21, 28 and 35, PM14-treated animals had a median (IQR) tumor volume of 401.2 (374.1 to 450.0), 472.7 (412.0 to 597.2), 743.8 (550.1 to 940.6), 1392 (1069 to 2085) and 2015 (1574 to 2161) mm³, respectively. Compared to placebo, PM14-treated animals experienced a high, statistically significant tumor reduction from days 7 to 14, that being the last measurement time in the euthanized, placebo-treated group, as shown in Table 5 below.

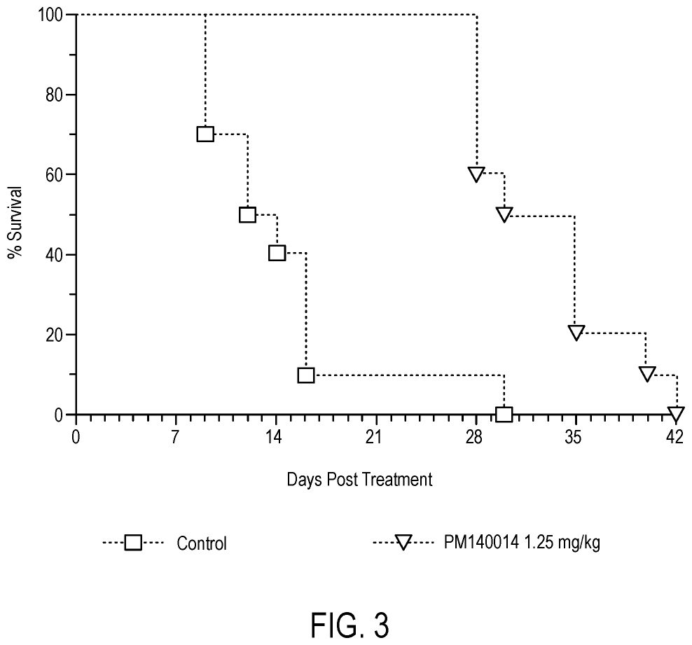

The survival time in the PM14-treated group was 32.5 days. The PM14 treatment statistically significantly increased the survival time compared to placebo (median survival time 13 days); p=0.0001), as shown in Table 6 below and in FIG. 3.

TABLE 5
Tumour volume (TV) obtained in mice bearing MRI-H-121 xenografts
and treated with Placebo or PM14 administered at 1.25 mg/kg.

| Compound | Day | TV, mm³ median (IQR) | | P (t-test) |
|---|---|---|---|---|
| Placebo | 0 | 190.0 | (162.7 to 220.0) | — |
| | 7 | 1147 | (956.4 to 1468) | — |
| | 14 | 1727 | (1228 to 1955) | — |
| PM14 | 0 | 190.8 | (168.8 to 217.4) | — |
| | 7 | 401.2 | (374.1 to 450.0) | 0.0002^{a} |
| | 14 | 472.7 | (412.0 to 597.2) | 0.0007^{a} |
| | 21 | 743.8 | (550.1 to 940.6) | N.P. |
| | 28 | 1392 | (1069 to 2085) | N.P. |
| | 35 | 2015 | (1574 to 2161) | N.P. |

^{a}Compared to placebo treated group.
N.P. No placebo group to compare with.

TABLE 6
Survival and statistic results obtained for mice
bearing MRI-H-121 xenografts and treated with
Placebo or PM14 administered at 1.25 mg/kg.

| Compound | Median survival time (days) | P |
|---|---|---|
| Placebo | 13 | — |
| PM14 | 32.5 | 0.0001^{a} |

^{a}Compared to placebo treated group.

In conclusion, PM14 displayed a good tolerability profiling in athymic mice bearing MRI-H-121 xenografted tumors.

Compared to placebo, PM14 treatment of mice bearing MRI-H-121 xenografts resulted in a high statistically significant (p<0.0007) reduction of tumor volume, as well as a high statistically significant increase in the survival time (p=0.0001) of PM14-treated animals.

### Example 4: Melanoma Activity in MEXF 276L, MEXL 462NL and MEXL 1341 L In Vitro Assays

IC₅₀ values were determined as described in Example 2, but using the following human cancer cell lines:
MEXF 276L (melanoma)
MEXF 462NL (melanoma)
MEXF 1341L (melanoma)

Results: The IC₅₀ values for MEXF 276L, MEXL 462NL and MEXL 1341L are shown below in Table 7, whilst the IC₇₀ values for MEXF 276L, MEXL 462NL and MEXL 1341 L are shown below in Table 8.

TABLE 7
IC₅₀ values for MEXF 276L, MEXL 462NL and MEXL 1341L

| | IC₅₀, μM (geometric mean) |
|---|---|
| Cell line | PM14 |
| MEXF 276L | 0.0002 |
| MEXL 462NL | 0.0001 |
| MEXL 1341L | 0.0004 |

TABLE 8
IC₇₀ values for MEXF 276L, MEXL 462NL and MEXL 1341L

| | IC₇₀, μM (geometric mean) |
|---|---|
| Cell line | PM14 |
| MEXF 276L | 0.0003 |
| MEXL 462NL | 0.0001 |
| MEXL 1341L | 0.0006 |

### Example 5: Melanoma Activity in WM-266-4 In Vitro Assay

GI₅₀ values were determined as described in Example 1, but using the following human cancer cell line:
WM-266-4 (ATCC@ CRL-1676) (Melanoma)

Cell line was obtained from the American Type Culture Collection (ATCC). Cells were maintained in MEM mediumCulture medium was supplemented with 10% Fetal Bovine Serum, 1% penicillin and streptomycin and 2 mM L-Glutamine. Cells were cultured at 37° C. and 5% CO₂ and kept always in a low-passage state.

Results: The GI₅₀ values for WM-266-4 are shown below in Table 9.

TABLE 9
GI₅₀ values for WM-266-4

| | GI₅₀, M |
|---|---|
| Cell line | PM14 |
| WM-266-4 | 1.94E−09 |

### Example 6: Simulations of PM14 Pharmacokinetics with Different Doses and Infusion-Rates

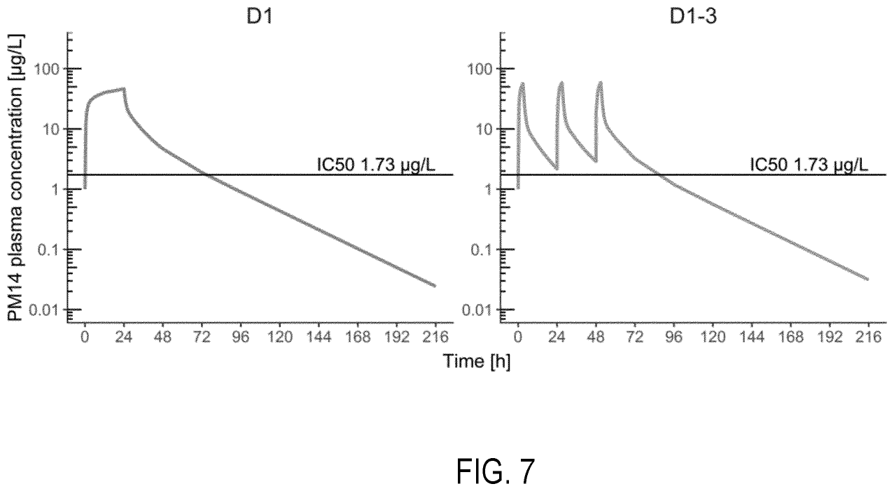

Simulations of PM14 pharmacokinetics with different doses and infusion-rates are shown in FIG. 7. The D1 simulation (Ihs) simulates 4.5 mg/m² with a 24 hour infusion. The D1-3 simulation (rhs) simulates ×3 1.5 mg/m² 3 hour infusions. The D1-3 schedule simulates a prolonged half-life without exceeding the 100 nM concentration.

### Example 7: Phase I, Open-Label, Dose-Escalating, Clinical and Pharmacokinetic Study of PM14 Administered Intravenously to Patients with Advanced Solid Tumors

Primary Study Objectives
Dose escalation phase: To identify the dose limiting toxicities (DLTs), and to determine the maximum tolerated dose (MTD) and the recommended dose (RD) of PM14 administered intravenously (i.v.) on two days (Day 1 and Day 8) or on Day 1 only, both every three weeks (q3wk) over three hours to patients with advanced solid tumors.

Secondary Study Objectives
To evaluate the safety and tolerability of PM14 i.v. given on Days 1 and 8 or on Day 1, both q3wk over three hours to patients with advanced solid tumors.
To determine the pharmacokinetics (PK) of PM14.
To evaluate pharmacogenetics (PGt) in germline DNA by the presence or absence of PGt polymorphisms in genes relevant for PM14 disposition (distribution, metabolism and excretion) that may explain individual variability in main PK parameters.
To conduct an exploratory pharmacogenomics (PGx) analysis in tumor tissue samples and circulating tumor DNA (ctDNA) of patients treated with PM14.
Dose escalation phase: To obtain information of the antitumor activity of PM14.

Study Design

First-in-human, open-label, dose-finding, phase I trial, using a classical 3+3 design followed by a continual reassessment method (CRM) (see Dose Escalation Schedule, below).

Patients will be included in cohorts of a minimum of three or six patients to receive PM14 at successively increasing dose levels, starting at 0.25 mg/m2 for the Days 1 and 8 schedule. For the Day 1 schedule, the starting dose will be 4.5 mg/m^{2.}

Dose escalation will proceed only after all the patients fully evaluable for DLT included at one dose level have completed the first cycle (i.e., three weeks).

According to the toxicities observed and pharmacokinetic results, other duration of infusions and/or schedules may be explored if considered appropriate, after agreement between the Sponsor, the Independent Monitoring Committee (IMC) and the Investigators.

Patients will receive PM14 until progression, unacceptable toxicity, consent withdrawal or while it is considered to be in their best interest. Radiological tumor assessments (and serum tumor markers, when appropriate) will be done every two cycles from treatment start until Cycle 6, and thereafter every three cycles while on treatment. After treatment discontinuation, patients will be followed until resolution or stabilization of all toxicities, if any. Patients discontinuing treatment without progression will be followed every three months until disease progression, start of other antitumor therapy, death or the end-of-study date (clinical cutoff: six months after treatment discontinuation of the last patient [last patient-last visit] or nine months after accrual of the last evaluable patient, whichever occurs first), whichever occurs first. After disease progression or start of a new therapy, patients will be followed up for survival every three months (±two weeks) until death or end-of-study date, whichever occurs first (a phone contact will be acceptable).

Antitumor response will be assessed using the RECIST v.1.1 and/or serum tumor markers as appropriate (see above).

Inclusion criteria:
1. Voluntarily signed and dated written informed consent (IC), obtained prior to any specific study procedure.
2. Age >18 years.
3. Eastern Cooperative Oncology Group (ECOG) performance status (PS) s 1.
4. For the Dose Escalation phase: Patients with pathologically confirmed diagnosis of advanced solid tumors for whom no curative standard therapy exists.
5. Life expectancy >3 months.
6. Patients with measurable or non-measurable disease according to the RECIST v.1.1 are eligible during the dose escalation phase.
7. Recovery to grade ≤1 from drug-related adverse events (AEs) of previous treatments, excluding alopecia and grade 1/2 asthenia or fatigue, according to the National Cancer Institute Common Terminology Criteria for Adverse Events (NCI-CTCAE v.4).
8. Laboratory values within seven days prior to first infusion:
   a) Absolute neutrophil count (ANC) 21.5×109/L, platelet count 100×109/L and hemoglobin >9 g/dL (patients may be transfused for anemia as clinically indicated prior to study entry).
   b) Aspartate aminotransferase (AST) and alanine aminotransferase (ALT) 53.0×upper limit of normal (ULN).
   c) Total bilirubin <ULN (up to 1.5×ULN for patients with Gilbert's syndrome). d) Creatinine clearance >30 mL/min (calculated using the Cockcroft and Gault's formula).
   e) Serum albumin >3 g/dL.
9. Wash-out periods:
   a) At least three weeks since the last chemotherapy (six weeks if therapy included nitrosoureas or systemic mitomycin C).
   b) At least four weeks since the last monoclonal antibody (MAb)-containing therapy or curative radiotherapy (RT).
   c) At least two weeks since the last biological/investigational single-agent therapy (excluding MAbs) and/or palliative RT (≤10 fractions or ≤30 Gy total dose).
   d) In patients with hormone-sensitive breast cancer progressing while on hormone therapy (except for luteinizing hormone-releasing hormone (LHRH) analogues in pre-menopausal women or megestrol acetate), all other hormonal therapies must be stopped at least one week before study treatment start.
   e) Castrate-resistant prostate cancer (CRPC) patients may continue receiving hormone therapy prior to and during study treatment.

Exclusion criteria:
1. Concomitant diseases/conditions:
   a) Increased cardiac risk:
      Uncontrolled arterial hypertension despite optimal management (>160/100 mmHg).
      Presence of clinically relevant valvular disease.
      History of long QT syndrome.
      Corrected QT interval (QTcF, Fridericia correction) >450 msec on screening electrocardiogram (ECG).
      History of ischemic heart disease, including myocardial infarction, angina, coronary arteriography or cardiac stress testing with findings consistent with coronary occlusion or infarction 56 months prior to study entry.
      History of heart failure or left ventricular dysfunction (left ventricular ejection fraction [LVEF] below normal values) by multiple-gated acquisition scan (MUGA) or echocardiography (ECHO).
      ECG abnormalities, including any of the following: left bundle branch block, right bundle branch block with left anterior hemiblock, second (Mobitz 1l) or third degree atrioventricular block.
      Symptomatic arrhythmia (excluding anemia-related sinusal tachycardia grade <2) or any arrhythmia requiring ongoing treatment, and/or prolonged QT-QTc grade >2; or presence of unstable atrial fibrillation. Patients with stable atrial fibrillation on treatment are allowed provided they do not meet any other cardiac or prohibited drug exclusion criterion.
      Clinically significant resting bradycardia (<50 beats per minute).
      Concomitant medication with risk of inducing torsades de pointes, which cannot be discontinued or switched to an alternative drug prior to start PM14 dosing.
      Use of a cardiac pacemaker.
   b) Active infection requiring systemic treatment.
   c) Known human immunodeficiency virus (HIV) or known hepatitis C virus (HCV) infection or active hepatitis B.
   d) Any other major illness that, in the Investigator's judgment, will substantially increase the risk associated with the patient's participation in this study (e.g., COVID-19).
2. Symptomatic, high dose steroid-requiring, and progressing central nervous system (CNS) disease. Exceptions will be made for (i) patients who have completed radiotherapy at least four weeks prior to inclusion (asymptomatic, non-progressing patients taking steroids in the process of already being tapered within two weeks prior to inclusion), and (ii) patients with asymptomatic brain metastasis without need for radiotherapy or steroids.
3. Patients with carcinomatous meningitis regardless of clinical stability.
4. Prior bone marrow or stem cell transplantation, or radiation therapy in more than 35% of bone marrow.
5. Prior treatment with trabectedin or lurbinectedin (PM01183) within six months prior to onset of study treatment.
6. Known hypersensitivity to any of the components of the drug product.
7. Limitation of the patient's ability to comply with the treatment or to follow the protocol procedures.
8. Pregnant or lactating women.

Women of childbearing potential (WOCBP) must agree to use an effective contraception method to avoid pregnancy during trial treatment and for at least six months after the last infusion. Fertile male patients must agree to refrain from fathering a child or donating sperm and to use an effective contraception method during treatment and for four months after the last infusion. WOCBP who are partners of fertile male patients must use an effective contraception method during the patients' treatment and for four months after the last infusion.

Expected Number of Patients

The number of patients may vary depending both on the tolerability to PM14 and the number of dose levels required to identify the MTD and the RD. Approximately 50 patients are expected to be recruited during dose escalation at three medical centers.

Methods

Open-label, dose-escalating, phase I trial of PM14 administered as a 3-hour infusion i.v. every 3 weeks (q3wk) in human patients (pts) with advanced solid tumors, adequate organ function and ECOG PS score of 0-1. Two schedules were explored: Schedule A (Day 1 [D1], Day 8 [D8]) and Schedule B (D1).

Evaluation Criteria

Primary Endpoints

Dose escalation phase:

Determination of MTD and RD: the MTD will be the lowest dose level explored during dose escalation in which one third or more of evaluable patients develop a DLT in Cycle 1. The CRM could be used to define the RD.

This protocol follows European terminology and thus the RD and the MTD are not equivalent.

Secondary Endpoints
Safety: patients will be evaluable for safety if they have received at least one partial infusion of PM14. AEs will be graded according to the NCI-CTCAE v.4. Additionally, treatment-related discontinuations and treatment compliance (dose reduction, skipped doses and/or treatment delays due to AEs), will be described.
Pharmacokinetics: PK analyses will be evaluated in plasma and urine by standard non-compartmental analysis (compartmental modeling may be performed if appropriate). Plasma samples for PM14 PK analysis will be obtained in Cycle 1 from all patients, and also in Cycle 2 from patients treated during Step D of the CRM. In addition, the urine produced during Day 1 of Cycle 1 and Cycle 2 will be collected from patients treated during Step D of the CRM.
Pharmacogenetics: the presence or absence of PGt polymorphisms in genes relevant for PM14 disposition (distribution, metabolism and excretion) from a single blood sample collected at any time during the trial (but preferably at the same time as the pre-treatment PK sample on Day 1 of Cycle 1) will be assessed to explain individual variability in main PK parameters.
Pharmacogenomics: this exploratory analysis will be performed in those patients who sign the Informed Consent Form (ICF) for the PGx study. mRNA or protein expression levels of factors involved in DNA repair mechanisms, or related to the mechanism of action of PM14, will be evaluated from available tumor tissue samples obtained at diagnosis or relapse and in cell-free ctDNA. Their mutational status might also be analyzed, if relevant. Their correlation with the clinical response and outcome after treatment will be assessed.
Efficacy: patients will be evaluable for efficacy if they receive at least one complete infusion of PM14 and have at least one clinical or radiological tumor assessment as per RECIST v.1.1 or serum markers, or if they are considered to have failed treatment. Treatment failure will be defined as clinical deterioration, death due to PD or treatment discontinuation due to any treatment-related toxicity before any appropriate tumor assessments have been performed.

Antitumor activity will be evaluated according to the RECIST v.1.1 and/or serum markers every two cycles (±one week) after treatment start in all patients with evaluable disease until Cycle 6. Those patients continuing treatment after Cycle 6 will thereafter have assessments performed every three cycles (±one week) while on treatment, unless otherwise is clinically indicated. Anonymized copies of all images must be submitted to the Sponsor.

Patients discontinuing treatment without progression will be followed every three months until disease progression, start of other antitumor therapy, death or the end-of-study date (clinical cutoff), whichever occurs first. After disease progression or start of new therapy, patients will be followed up for survival every three months (±two weeks) until death or until the end-of-study date, whichever occurs first (a phone contact will be acceptable).

Efficacy endpoints comprise response rates (percentage of patients with PR, with CR, or the sum of both [ORR]), percentage of patients with stable disease (SD) 4 months, percentage of patients with clinical benefit (ORR or SD 4 months), and time-to-event parameters (if appropriate). Efficacy endpoints will be secondary endpoints.

Dose Limiting Toxicity Definition

DLTs are defined as AEs and laboratory abnormalities related to the study drug occurring during the first cycle of treatment and fulfilling at least one of the criteria outlined below:
Grade 4 neutropenia (ANC <0.5 ×109/L) lasting ≥3 days.
Febrile neutropenia or neutropenic sepsis of any duration.
Grade 4 thrombocytopenia (platelet count <25×109/L) or grade 3 thrombocytopenia with bleeding requiring a platelet transfusion.
Grade 4 ALT or AST increase or grade 3 increase lasting >7 days.
Grade ≥2 ALT or AST increase concomitantly with total bilirubin increase >2.0×ULN and normal alkaline phosphatase (ALP) (i.e., fulfilling Hy's Law criteria).
Any other grade 3/4 non-hematological AE related to the study drug, with the following exceptions:
   1) Nausea/vomiting (unless receiving standard antiemetic treatment).
   2) Grade 3 diarrhea lasting less than two days (unless receiving standard treatment).
   3) Grade 3 asthenia lasting less than one week.
   4) Hypersensitivity reactions.
   5) Extravasations.
   6) Non-clinically relevant biochemical abnormalities (e.g., isolated increase in gamma-glutamyltransferase [GGT]). In any case, the clinical relevance should be discussed.
Only for the Days 1 and 8 schedule, failure to administer the Day 8 infusion (with a treatment window of +72 hours) due to AE(s) related to the study drug in Cycle 1.
Delay in the administration of the second cycle of PM14 exceeding 14 days, due to AE(s) related to the study drug.
The following circumstances should be discussed and the final consensus should be documented:
   DLTs with delayed onset (i.e. after the end of Cycle 1).
   Non-compliance with the intended dose intensity or frequent dose delays or omissions due to AE(s) related to the study drug.

Replacement of Patients

Dose escalation phase: Patients must be replaced if they are not fully evaluable for the assessment of the primary objective (determination of the MTD and RD).

An evaluable patient for the main objective of the phase must have received at least one complete cycle, except if the discontinuation, missed dose, delay or interruption were due to toxicity, and must have been adequately followed during Cycle 1 (three weeks).

Specifically, patients must be replaced if:
They are withdrawn from the study before completing a PM14 cycle (Day 1 and Day 8 infusions for the two infusion schedule plus two resting weeks; or Day 1 for the one infusion schedule; plus three resting weeks) for any reason other than toxicity (excluding hypersensitivity and/or extravasation reactions).
They receive any forbidden concomitant medication or have other therapeutic procedure (i.e., major surgery) within three weeks after the first dose, unless they previously had a DLT.
There is a protocol deviation resulting in the impossibility to obtain any conclusion regarding safety during Cycle 1.

Criteria for Treatment Continuation

Patients may be treated with additional cycles of PM14 as long as no unacceptable toxicity and/or progression of the disease occurred. Criteria for treatment continuation are included in Table 10 and Table 11.

The administration of a new cycle should be delayed if these criteria are unmet on the corresponding Day 1 of each cycle. Parameters will be re-evaluated after at least 48 hours, or more if appropriate. The new cycle will always start only upon recovery of these parameters. A maximum delay of 14 days will be allowed for recovery from any drug-related AEs. Should recovery not occur after this period, the patient must discontinue treatment except in case of objective patient clinical benefit according to the Investigator's criteria and request, and upon the Sponsor's approval.

Only for Day 1 and 8 schedule, if treatment continuation criteria are not met on Day 8 of any cycle, the scheduled Day 8 infusion will be withheld for a maximum of 72 hours; if after this period the criteria are still not met, the scheduled Day 8 infusion will be skipped. Only infusions scheduled on Day 1 can be delayed.

TABLE 10
Criteria for Treatment Continuation: Days 1 and 8

| | | |
|---|---|---|
| Variable | Day 1 | Day 8 ^{a} |

| | | | | |
|---|---|---|---|---|
| Hemoglobin | ≥9 | g/dL | ≥9 | g/dL |

| | | |
|---|---|---|
| ANC | ≥1.5 × 10⁹/L | ≥1.0 × 10⁹/L |
| Platelets | ≥100 × 10⁹/L | ≥80 × 10⁹/L |

| | | | | |
|---|---|---|---|---|
| Creatinine clearance | ≥30 | mL/min ^{b} | ≥30 | mL/min ^{b} |

| | | |
|---|---|---|
| Total bilirubin | ≤ULN ^{c} | ≤ULN ^{c} |
| AST, ALT | ≤3.0 × ULN | ≤3.0 × ULN |
| Other non- hematological | Grade ≤1 or to | Grade ≤1 or to |
| treatment-related AEs ^{d} | baseline values | baseline values |

^{a} Only infusions scheduled on Day 1 can be delayed. If treatment continuation criteria are not met on Day 8 of any cycle, the scheduled Day 8 infusion will be withheld for a maximum of 72 hours; if after this period the criteria are still not met, the scheduled Day 8 infusion will be skipped.
^{b} Calculated using the Cockcroft and Gault's formula.
^{c} Up to 1.5 × ULN for patients with Gilbert's syndrome.
^{d} Except for alopecia and/or vomiting in patients who are not receiving optimal antiemetic medications, or non-clinically relevant laboratory abnormalities, e.g. isolated increase in GGT.
AEs, adverse events; ALT, alanine aminotransferase; ANC, absolute neutrophil count; AST, aspartate aminotransferase; GGT, gamma-glutamyltransferase; ULN, upper limit of normal.

The decision of continuing treating patients with skipped doses will be evaluated on a case-by-case approach and after agreement between the Investigator and the Sponsor.

TABLE 11
Criteria for Treatment Continuation: Day 1 schedule.

| Variable | Day 1 | |
|---|---|---|
| Hemoglobin | ≥9 | g/dL |
| ANC | ≥1.5 × 10⁹/L | |
| Platelets | ≥100 × 10⁹/L | |
| Creatinine clearance | ≥30 | mL/min ^{a} |

| | |
|---|---|
| Total bilirubin | ≤ULN ^{b} |
| AST, ALT | ≤3.0 × ULN |
| Other non-hematological | Grade <1 or to |
| treatment-related AEs ^{c} | baseline values |

^{a} Calculated using the Cockcroft and Gault's formula.
^{b} Up to 1.5 × ULN for patients with Gilbert's syndrome.
^{c} Except for alopecia and/or vomiting in patients who are not receiving optimal antiemetic medications, or non-clinically relevant laboratory abnormalities, e.g. isolated increase in GGT.
AEs, adverse events; ALT, alanine aminotransferase; ANC, absolute neutrophil count; AST, aspartate aminotransferase; GGT, gamma-glutamyltransferase; ULN, upper limit of normal.

Dose Reduction

Treatment after DLT, a treatment-related infusion delay longer than 14 days, or any treatment-related AE considered as unacceptable by the Investigators may continue only if there is clear evidence of objective patient's clinical benefit. This will always be discussed with the Sponsor. Under these circumstances, and always following recovery to pre-specified re-treatment criteria, patients will receive a subsequent infusion at the dose level immediately below the one administered during the previous infusion during dose escalation (i.e., Steps A, B and C).

If dose reductions are required at the starting dose or at dose level 2, a decision about study continuation and the following dose to be administered to the patients affected will be discussed between the Sponsor and the Investigators. Patients requiring a dose reduction during Step D due to the aforementioned circumstances will receive a subsequent infusion at a dose level 20% lower than the one administered during the previous infusion.

Up to two individual dose reductions will be allowed per patient; any patients requiring more than two dose reductions will discontinue treatment. Once the dose has been reduced for an individual patient, it will not be re-escalated again.

Results

The results of the dose escalation study are as follows:

Patient Characteristics

The patient characteristics are summarised below in Table 12:

TABLE 12
Summary of patients tested under Schedule A and Schedule B

| | A | B |
|---|---|---|
| | D1, D8 q3wk | D1 q3wk |
| | Schedule (n: 28) | Schedule (n: 9) |
| Age (range) | 56 (23-78) | 47 (36-76) |
| Gender (M/F) | 16 (57.1%)/12 (42.9%) | 5 (55.6%)/4 (44.4%) |
| ECOG 0 | 16 (57.1%)/12 (42.9%) | 5 (55.6%)/4 (44.4%) |
| Most Common | Soft tissue sarcoma (n: 6) | Colorectal (n: 2) |
| Tumor Types | Ovarian cancer (n: 5) | |
| | Pancreas (n: 3) | |
| | Prostate (n: 3) | |
| No. Sites of Disease | 4 (1-8) | 2 (1-6) |
| (range) | | |
| No. Prior Lines (range) | 3 (1-8) | 4 (1-8) |

Results: 37 pts were treated (Schedule A/B: 28/9 pts). Baseline characteristics of pts (A/B): median age 56/47 years; male 57%/56%; ECOG PS 0: 57%/56%; median of prior lines (range): 3 (1-8)/4 (1-10). Most common tumor types (A +B): STS (n=7 pts), ovarian (n=6), pancreatic (n=4), prostate cancer (n=3). The maximum tolerated dose was 4.5 mg/m² for A (dose-limiting toxicities [DLTs]: D8 omission due to lack of recovery of lab parameters for re-treatment [n=2 pts]) and 5.6 mg/rn² (DLTs: G4 febrile neutropenia [n=1], G4 transaminase increase [n=1]) for B.

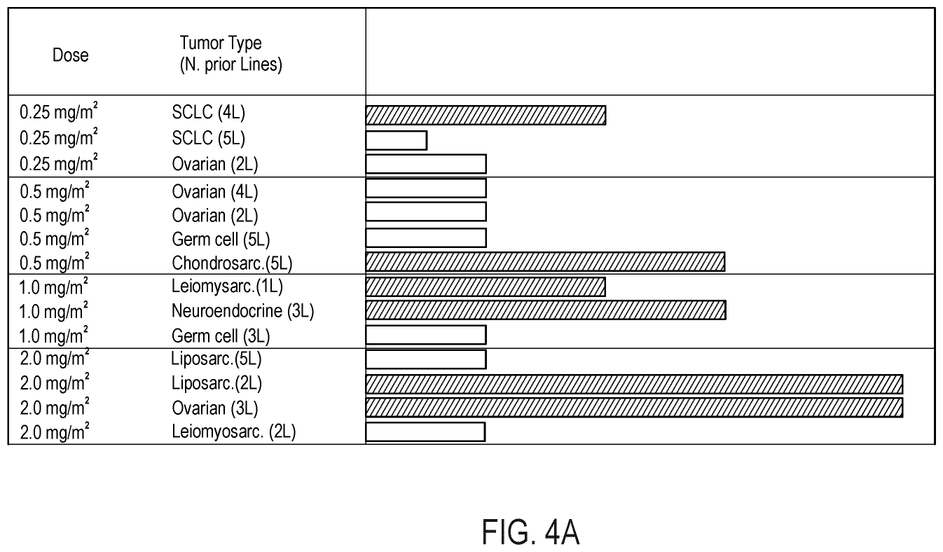

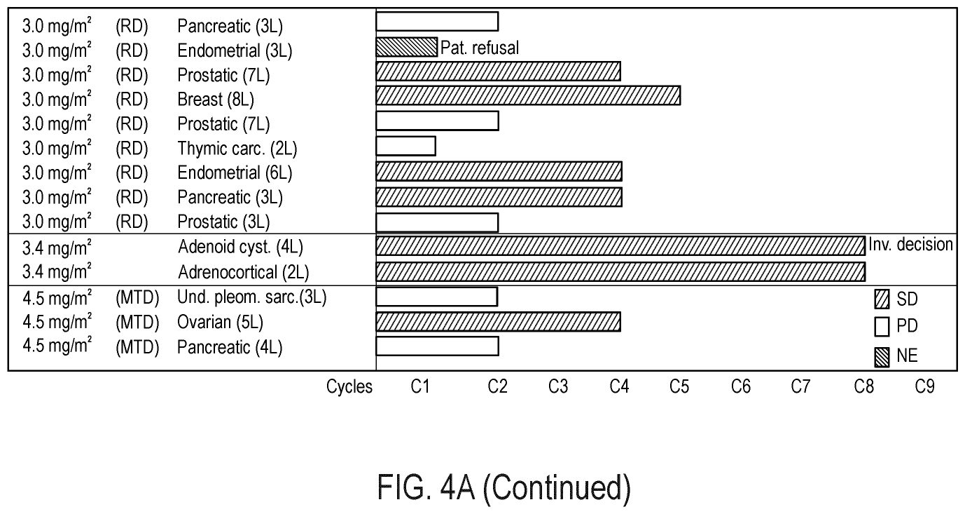

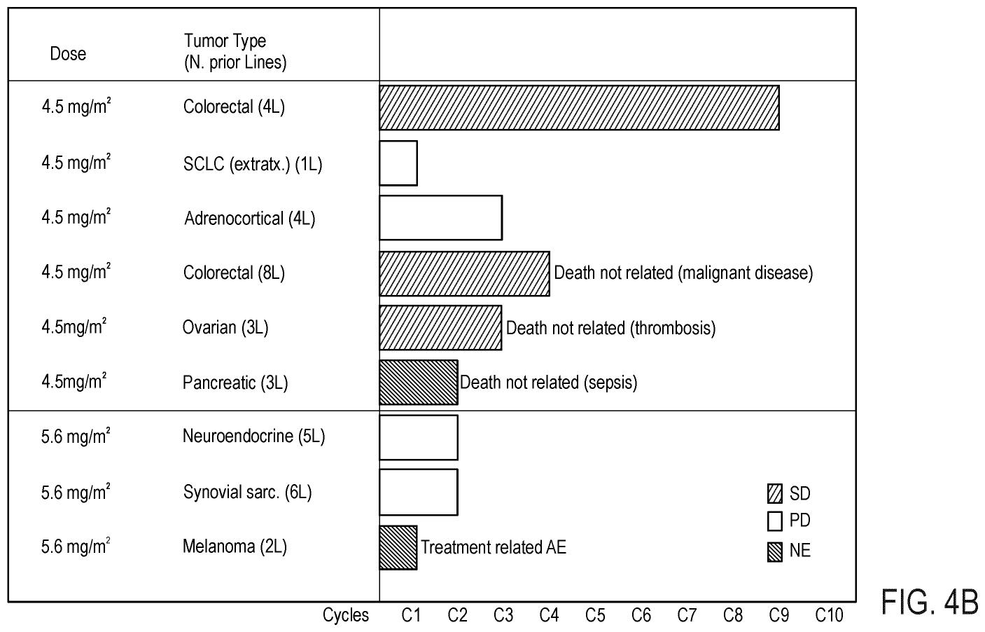

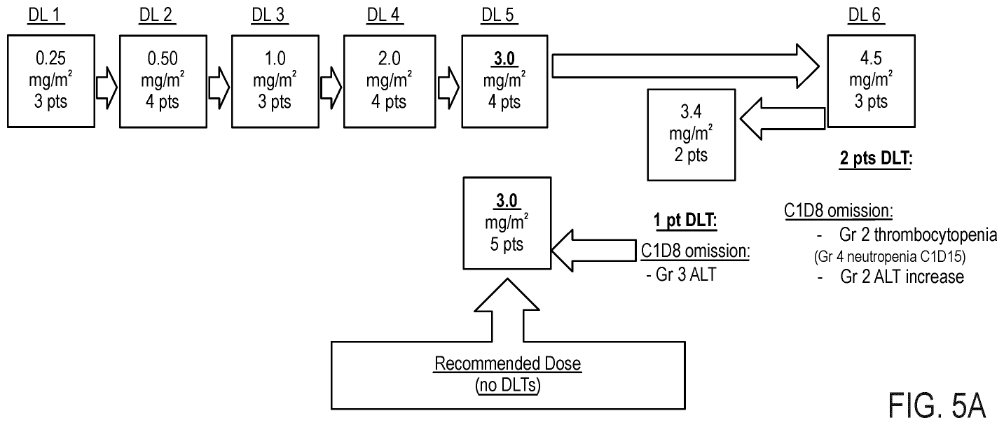

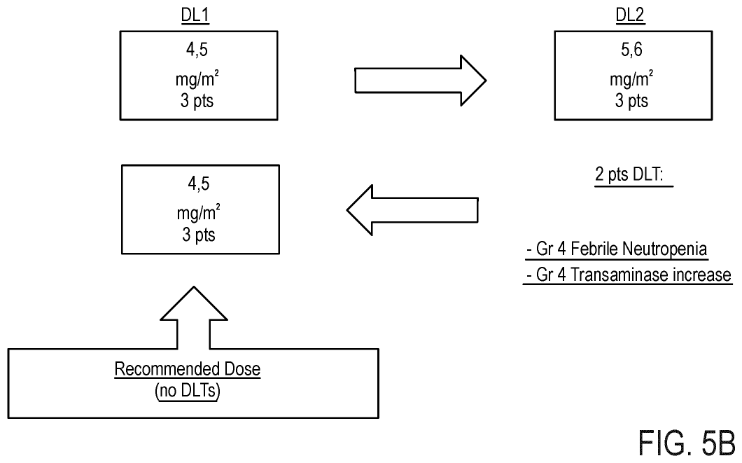

The recommended dose (RD) was 3.0 mg/n² on D1,D8 (A), and 4.5 mg/n² on D1(B). No DL-Ts were present at the RDs. Most common toxicities were hematological abnormalities and transaminase increase. The efficacy results are shown in FIGS. 4A and 41B, whilst the safety results are shown in Tables 13 and 14 below and summarised in FIGS. 5A and 5B.

TABLE 13
Most common related (or UNK) AES or laboratory abnormalities
for D 1, D 8 schedule (Schedule A)

| | <3.0 mg/m² | | 3.0 mg/m² | | | >3.0 mg/m² | | |
|---|---|---|---|---|---|---|---|---|
| | (n: 14) | | (n: 9) | | | (n: 5) | | |
| | Gr 1-2 | Gr 3 | Gr 1-2 | Gr 3 | Gr 4 | Gr 1-2 | Gr 3 | Gr 4 |
| | n (%) | n (%) | n (%) | n (%) | n (%) | n (%) | n (%) | n (%) |
| Anemia | 12 | — | 7 | 1 | — | 4 | 1 | — |
| | (85.7%) | | (77.8%) | (11.1%) | | (80.0%) | (20.0%) | |
| Neutropenia | 2 | — | 1 | 1 | 1 | 1 | — | 2 |
| | (14.3%) | | (11.1%) | (11.1%) | (11.1%) | (20.0%) | | (40.0%) |
| Febrile | — | — | — | — | — | — | — | — |
| neutropenia | | | | | | | | |
| Thrombo- | 2 | — | 1 | — | — | 2 | — | 1 |
| cytopenia | (14.3%) | | (11.1%) | | | (40.0%) | | (20.0%) |
| ALT | 5 | — | 5 | 1 | — | 3 | 2 | — |
| increase | (35.7%) | | (55.6%) | (11.1%) | | (60.0%) | (40.0%) | |
| AST | 3 | — | 7 | — | — | 5 | — | — |
| increase | (21.4%) | | (77.8%) | | | (100.0%) | | |
| CPK | 6 | — | 2 | — | — | — | — | — |
| increase | (42.9%) | | (22.2%) | | | | | |
| Fatigue | 4 | — | — | — | — | 2 | 1 | — |
| | (28.6%) | | | | | (40.0%) | (20.0%) | |
| Nausea | 6 | — | 1 | — | — | 4 | — | — |
| | (42.9%) | | (11.1%) | | | (80.0%) | | |
| Vomiting | 6 | — | 1 | 1 | — | 3 | — | — |
| | (42.9%) | | (11.1%) | (11.1%) | | (60.0%) | | |
| Decreased | 1 | — | — | — | — | 2 | — | — |
| appetite | (7.1%) | | | | | (40.0%) | | |
| Diarrhoea | 2 | — | 1 | — | — | 1 | — | — |
| | (14.3%) | | (11.1%) | | | (20.0%) | | |
| Constipation | 1 | — | — | — | — | 3 | — | — |
| | (7.1%) | | | | | (60.0%) | | |

TABLE 14
Most common related (or UNK) AES or laboratory
abnormalities for D 1 schedule (Schedule B)

| | 4.5 mg/m² | | | 5.6 mg/m² | | |
|---|---|---|---|---|---|---|
| | (n: 6) | | | (n: 3) | | |
| | Gr 1-2 | Gr 3 | Gr 4 | Gr 1-2 | Gr 3 | Gr 4 |
| | n (%) | n (%) | n (%) | n (%) | n (%) | n (%) |
| Anemia | 5 | — | — | 1 | 2 | — |
| | (83.3%) | | | (33.3%) | (66.7%) | |
| Neutropenia | — | 1 | 1 | 1 | 1 | 1 |
| | | (16.7%) | (16.7%) | (33.3%) | (33.3%) | (33.3%) |
| Febrile | — | — | — | — | — | 1 |
| neutropenia | | | | | | (33.3%) |
| Thrombo- | — | 2 | — | 2 | 1 | — |
| cytopenia | | (33.3%) | | (66.7%) | (33.3%) | |
| ALT | 4 | 2 | — | 2 | — | 1 |
| increase | (66.7%) | (33.3%) | | (66.7%) | | (33.3%) |
| AST | 3 | — | 1 | 1 | — | 1 |
| increase | (50.0%) | | (16.1%) | (33.3%) | | (33.3%) |
| CPK | 2 | — | — | 2 | — | — |
| increase | (33.3%) | | | (66.7%) | | |
| Fatigue | 3 | — | — | 1 | — | — |
| | (50.0%) | | | (33.3%) | | |
| Nausea | 3 | — | — | — | — | — |
| | (50.0%) | | | | | |
| Vomiting | 1 | — | — | — | — | — |
| | (16.7%) | | | | | |
| Decreased | 1 | — | — | — | — | — |
| appetite | (16.7%) | | | | | |
| Headache | 1 | — | — | — | — | — |
| | (16.7%) | | | | | |

Pharmacokinetics

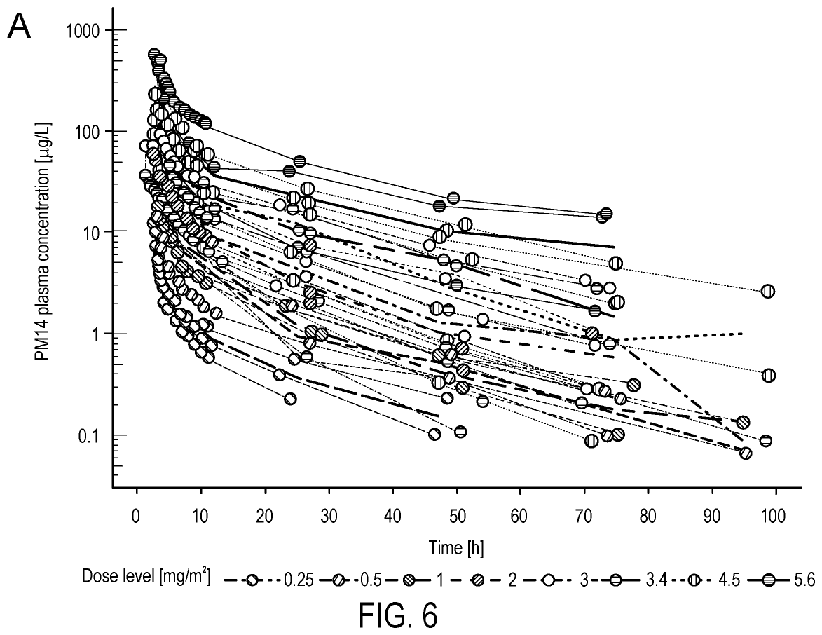

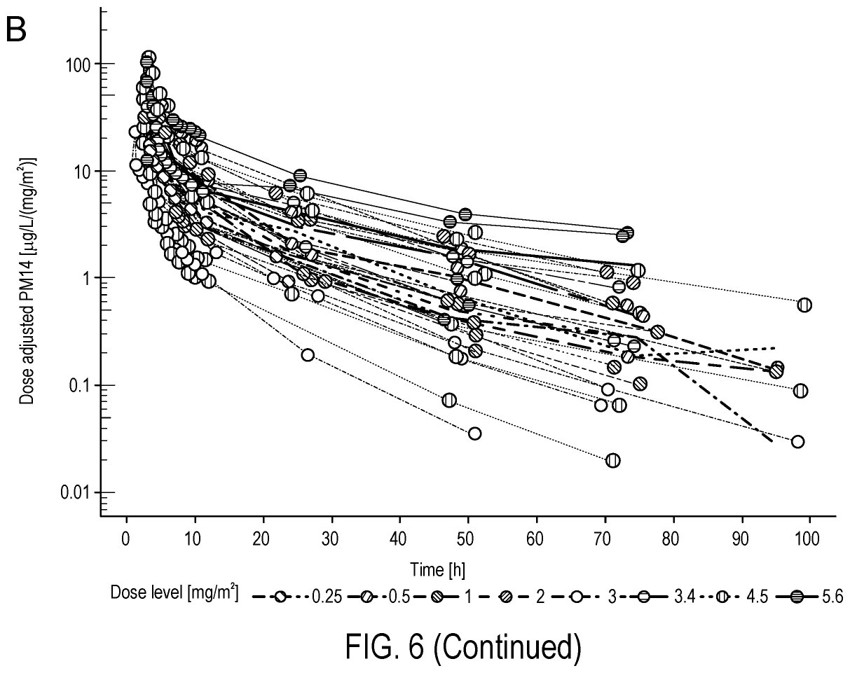

Linear pharmacokinetics were observed for PM14 at tested doses (0.25-5.6 mg/rn²), with geometric mean (CV %) total plasma clearance 5.9 L/h (88%), volume of distribution 128 L (81%) and median (range) terminal half-life 15.9 h (7.5-34.3 h). Less than 1.6% of administered dose was recovered in urine. The pharmacokinetic data is shown in FIGS. 6A and 6B.

The dose escalation study has determined RDs for two PM14 schedules in patients with advanced solid tumors. At the RDs, PM14 is well tolerated and has a manageable safety profile. The most common related adverse events are transient transaminase increase, nausea/vomiting, fatigue and neutropenia. Some prolonged tumor stabilizations were observed in patients, including heavily pre-treated patients with soft tissue sarcoma, epithelial ovarian cancer, colorectal cancer and adrenocortical carcinoma. PK of PM14 is linear at the range of doses tested, hepatic extraction ratio is low and distribution into peripheral tissues is moderate, resulting in a half-life of 16 h.

The study shows stable disease (SD) in respect of various cancers including: SCLC; STS, including leiomyosarcoma and liposarcoma; bone sarcoma including myxoid chondrosarcoma; neuroendocrine tumor; ovarian cancer; breast cancer; endometrial cancer; prostate cancer, pancreatic cancer; adenoid cystic carcinoma; adrenocortical carcinoma; and colorectal cancer.

Overall, the data in the present invention has demonstrated that PM14 can be used in the treatment of various cancers such as SCLC; sarcoma including STS and bone sarcoma; STS including leiomyosarcoma and liposarcoma; bone sarcoma including chondrosarcoma; melanoma including amelanotic melanoma, neuroendocrine tumor; ovarian cancer; breast cancer; endometrial cancer; pancreatic cancer; adenoid cystic carcinoma; adrenocortical carcinoma; renal cancer including renal carcinoma, kidney clear cell carcinoma, hypernephroma or poorly differentiated hypernephroma; and colorectal cancer. Separately, the present invention has also identified for the first time dosage regimens useful in the treatment of cancer. These dosage regimens have been determined to be well tolerated with a manageable safety profile. Evidence of efficacy in humans has also been demonstrated.

The cancer may be selected from lung cancer including non-small cell lung cancer and small cell lung cancer; colon cancer; rectal cancer; colorectal cancer; breast cancer; pancreas cancer; sarcoma including soft tissue sarcoma and bone sarcoma; soft tissue sarcoma including fibrosarcoma, leiomyosarcoma and liposarcoma; bone sarcoma including chondrosarcoma, or myxoid chondrosarcoma; ovarian cancer; prostate cancer; gastric cancer; renal cancer including renal carcinoma, kidney clear cell carcinoma, hypernephroma and poorly differentiated hypernephroma; melanoma, including amelanotic melanoma; neuroendocrine tumor; endometrial cancer; adenoid cystic carcinoma; and adrenocortical carcinoma.

Accordingly, the present invention provides new effective options for the treatment of cancer.

1. A compound of formula I: 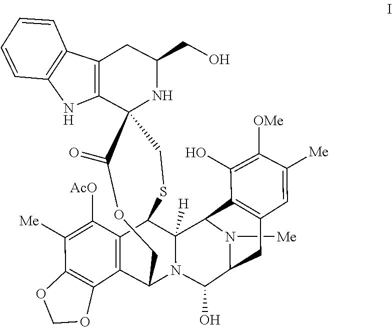 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]
or a pharmaceutically acceptable salt or ester thereof, for use in the treatment of cancer, wherein the compound is administered to a subject in a three-week cycle at a total dose from about 0.5 mg/m² to about 9 mg/m², preferably from about 1.0 mg/m² to about 9.0 mg/m², about 1.5 mg/m² to about 9.0 mg/m², about 2.0 mg/m² to about 9.0 mg/m², about 2.5 mg/m² to about 8.5 mg/m², about 3.0 mg/m² to about 8.0 mg/m2, about 3.5 mg/m² to about 7.5 mg/m², about 4.0 mg/m² to about 7.0 mg/m², about 4.0 mg/m² to about 6.5 mg/m², about 4.5 mg/m² to about 6.5 mg/m², about 4.5 mg/m² to about 6.0 mg/m^{2.}

2. A compound for use according to claim 1, wherein the total dose is about 3.0 mg/m² to about 6.0 mg/m², about 3.0 mg/m² to about 5.6 mg/m², about 3.5 mg/m² to about 5.6 mg/m², about 4.0 mg/m² to about 5.0 mg/m² or about 4.5 mg/m^{2.}

3. A compound for use according to claim 1, wherein the total dose is about 4.0 mg/m² to about 5.5 mg/m², about 4.5 mg/m² to about 5.0 mg/m², about 4.5 mg/m² or about 5.0 mg/m^{2.}

4. A compound for use according to claim 1, wherein the total dose is about 4.0 mg/m² to about 9.0 mg/m², about 4.0 mg/m² to about 8.0 mg/m², about 4.5 mg/m² to about 7.5 mg/m², about 5.0 mg/m² to about 7.0 mg/m², about 5.5 mg/m² to about 6.5 mg/m², or about 6.0 mg/m^{2.}

5. A compound for use according to claim 1, wherein the total dose is about 6.0 mg/m² to about 9.0 mg/m², more preferably about 6.5 mg/m² to about 8.5 mg/m², more preferably about 7.0 mg/m² to about 8.0 mg/m², more preferably 7.0 mg/m² or 8.0 mg/m^{2.}

6. A compound for use according to any one of claims 1 to 3, wherein the compound is administered as a single dose during the three-week cycle.

7. A compound for use according to claim 6, wherein the single dose is about 4.5 mg/m^{2.}

8. A compound for use according to claim 6, wherein the single dose is about 5.0 mg/m^{2.}

9. A compound for use according to claim 1, claim 4 or claim 5, wherein the compound is administered as a first dose and a second dose during the three-week cycle.

10. A compound for use according to claim 9, wherein the first dose is administered on day 1 of the three-week cycle and the second dose is administered on day 8 of the three-week cycle.

11. A compound for use according to any one of claims 9 to 10, wherein the amount of compound administered for the first dose and the amount of compound administered for the second dose are equal.

12. A compound for use according to any one of claims 9 to 11, wherein the total dose for the first dose and the second dose is 6.0 mg/m^{2.}

13. A compound for use according to any one of claims 9 to 11, wherein the total dose for the first dose and the second dose is 7.0 mg/m^{2.}

14. A compound for use according to any one of claims 9 to 11, wherein the total dose for the first dose and the second dose is 8.0 mg/m^{2.}

15. A compound for use according to any one of claims 9 to 11, wherein the first dose is 3.0 mg/m² and the second dose is 3.0 mg/m^{2.}

16. A compound for use according to any one of claims 9 to 11, wherein the first dose is 3.5 mg/m² and the second dose is 3.5 mg/m^{2.}

17. A compound for use according to any one of claims 9 to 11, wherein the first dose is 4.0 mg/m² and the second dose is 4.0 mg/m^{2.}

18. A compound for use according to claim 1, wherein the compound of formula I is administered 2, 3, 4, 5, 6, 7, 8, 9 or 10 times during the three-week cycle.

19. A compound for use according to claim 18, wherein the compound of formula I is administered 2 times during the three-week cycle.

20. A compound for use according to claim 18, wherein the compound of formula I is administered 3 times during the three-week cycle.

21. A compound for use according to claim 20, wherein the compound of formula I is administered on days 1, 2 and 3 of the three-week cycle.

22. A compound for use according to any one of claims 18 to 21, wherein the total dose is about 0.5 mg/m², about 1.0 mg/m², about 1.5 mg/m², about 2.0 mg/m², about 2.5 mg/m2, about 3.0 mg/m², about 3.5 mg/m², about 4.0 mg/m², about 4.5 mg/m², about 5.0 mg/m2, about 5.5 mg/m², about 6.0 mg/m², about 6.5 mg/m², about 7.0 mg/m², 7 about.5 mg/m2, about 8.0 mg/m², about 8.5 mg/m² or about 9.0 mg/m^{2.}

23. A compound for use according to any one of claims 18 to 22, wherein each individual dose (i.e. each day) is about 0.5 mg/m², 1.0 mg/m², 1.5 mg/m², 2.0 mg/m², 2.5 mg/m², 3.0 mg/m², 3.5 mg/m², 4.0 mg/m², 4.5 mg/m², 5.0 mg/m², 5.5 mg/m², 6.0 mg/m², 6.5 mg/m^{2, 7.0} mg/m², 7.5 mg/m², 8.0 mg/m², 8.5 mg/m² or 9.0 mg/m^{2.}

24. A compound for use according to any one of claims 1 to 23, wherein the compound is administered parentally, preferably intravenously.

25. A compound for use according to claim 24, wherein the compound is administered by infusion.

26. A compound for use according to claim 25, wherein the infusion time is up to 24 hours.

27. A compound for use according to claim 25, wherein the infusion time is 3 hours.

28. A compound for use according to claim 25, wherein the infusion time is 24 hours.

29. A compound for use according to any one of claims 1 to 28, wherein the compound is administered together with radiation therapy; said radiation therapy being administered prior to, concurrently with or subsequently to administration of the compound.

30. A compound for use according to any one of claims 1 to 29, wherein the cancer is selected from lung cancer including non-small cell lung cancer and small cell lung cancer, colon cancer, rectal cancer, colorectal cancer, breast cancer, pancreas cancer, sarcoma including soft tissue sarcoma or bone sarcoma, ovarian cancer, prostate cancer, gastric cancer, renal cancer, melanoma, neuroendocrine tumor, endometrial cancer, adenoid cystic carcinoma and adrenocortical carcinoma.

31. A compound for use according to claim 30, wherein the renal cancer is renal carcinoma, kidney clear cell carcinoma or hypernephroma, including poorly differentiated hypernephroma.

32. A compound for use according to claim 30, wherein the melanoma is amelanotic melanoma.

33. A compound for use according to claim 30, wherein the soft tissue sarcoma is selected from fibrosarcoma, leiomyosarcoma and liposarcoma.

34. A compound for use according to claim 30, wherein the bone sarcoma is chondrosarcoma, preferably myxoid chondrosarcoma.

35. A compound for use according to any one of claims 1 to 34, wherein the salt is selected from hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate, p-toluenesulfonate, sodium, potassium, calcium, ammonium, ethylenediamine, ethanolamine, N,N-dialkylenethanolamine, triethanolamine and basic amino acids.

36. A pharmaceutical composition comprising a compound of formula I or a pharmaceutically acceptable salt or ester thereof, and a pharmaceutically acceptable carrier, for use according to any one of claims 1 to 35.

37. A dosage form comprising a pharmaceutical composition according to claim 25, for use according to any one of claims 1 to 35.

38. Use of a compound according to any one of claims 1 to 35, or a pharmaceutically acceptable salt or ester thereof, or a pharmaceutical composition according to claim 36, or a dosage form according to claim 37, in the manufacture of a medicament for the treatment of a cancer, wherein the compound is administered as defined in any one of claims 1 to 35.

39. A method of treating a cancer in a patient in need thereof, comprising administering to the patient a therapeutically effective amount of a compound according to any one of claims 1 to 35, or a pharmaceutically acceptable salt or ester thereof, or a pharmaceutical composition according to claim 36, or a dosage form according to claim 37, wherein the compound is administered as defined in any one of claims 1 to 35.

40. A kit comprising a compound according to claim 1, together with instructions for administration in accordance with any one of claims 1 to 35.

41. A compound of formula I: 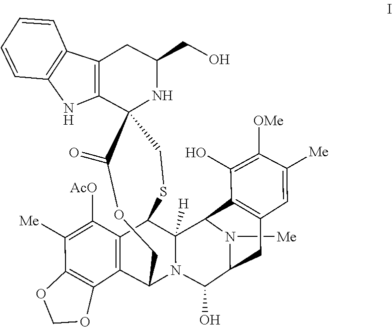 [SMILES: COc1c(C)cc2c(c1O)[C@@H]1[C@@H]3[C@@H]4SC[C@]5(N[C@H](CO)Cc6c5[nH]c5ccccc65)C(=O)OC[C@@H](c5c6c(c(C)c(OC(C)=O)c54)OCO6)N3[C@@H](O)[C@H](C2)N1C]
or a pharmaceutically acceptable salt or ester thereof, for use in the treatment of a cancer selected from renal cancer, melanoma, neuroendocrine tumor, endometrial cancer, adenoid cystic carcinoma, adrenocortical carcinoma, bone sarcoma and soft tissue sarcoma.

42. A compound for use according to claim 41, wherein the renal cancer is renal carcinoma, kidney clear cell carcinoma or hypernephroma, wherein the hypernephroma may be poorly differentiated hypernephroma.

43. A compound for use according to claim 41, wherein the melanoma is amelanotic melanoma.

44. A compound for use according to claim 41, wherein the soft tissue sarcoma is selected from leiomyosarcoma and liposarcoma.

45. A compound for use according to claim 41, wherein the bone sarcoma is chondrosarcoma, preferably myxoid chondrosarcoma.

46. A compound for use according to any one of claims 41 to 45, wherein the salt is selected from hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate, p-toluenesulfonate, sodium, potassium, calcium, ammonium, ethylenediamine, ethanolamine, N,N-dialkylenethanolamine, triethanolamine and basic amino acids.

47. A pharmaceutical composition comprising a compound of formula I or a pharmaceutically acceptable salt or ester thereof, and a pharmaceutically acceptable carrier, for use according to any one of claims 41 to 46.

48. A dosage form comprising a pharmaceutical composition according to claim 36, for use according to any one of claims 41 to 46.

49. Use of a compound according to any one of claims 41 to 46, or a pharmaceutically acceptable salt or ester thereof, or a pharmaceutical composition according to claim 47, or a dosage form according to claim 48, in the manufacture of a medicament for the treatment of a cancer as defined in any one of claims 41 to 46.

50. A method of treating a cancer in a patient in need thereof, comprising administering to the patient a therapeutically effective amount of a compound according to any one of claims 41 to 46, or a pharmaceutically acceptable salt or ester thereof, or a pharmaceutical composition according to claim 47, or a dosage form according to claim 48, wherein the cancer is as defined in any one of claims 41 to 46.

51. A kit comprising a compound according to claim 41, together with instructions for administration in accordance with any one of claims 41 to 50.
